(19) United States
(12) Patent Application Publication
(10) Pub. No.: US 2002/0058031 A1
(43) Pub. Date: May 16, 2002

(54) Methods for preparing diagnostic reagents using antibody preparation

(76) Inventors: Hsiaoho Edward Tung, San Diego, CA (US); Robert Thomas Hudak, Carlsbad, CA (US)

Correspondence Address: Jinn-Nan Lin, 4180 Sorrento Valley Boulevard, San Diego, CA, 92121, US

(21) Appl. No.: 09/957,145

(22) Filed: Sep. 17, 2001

Related U.S. Application Data
(60) Provisional application No. 60/233,739, filed on Sep. 19, 2000.

(51) Int. Cl. A61K 39/395; C12P 21/04; G01N 33/53; G01N 33/542

(52) U.S. Cl. 424/140.1; 435/70.21; 435/7.9; 530/388.1; 435/326

(57) ABSTRACT

The present invention utilizes human antibodies to identify and produce antigen preparations that are particularly useful for the detection, diagnosis, prognosis or prevention of human etiological agents or human disease states or conditions. The present invention provides a variety of aspects, including antigenic preparations, antibody preparations, methods of making such preparations and methods of using such preparations.

The present invention is:

a method for preparing a composition relating to a human etiological agent and/or a human diseased state or condition.

a method for detecting an antibody and/or an antigen that binds with an antigenic preparation relating to a human etiological agent and/or to a human diseased state or condition.

a composition

a method for antibody preparation; an antibody, an antibody preparation, a hybridoma or a cell.

a diagnostic tool, a test a region or a zone.

THIS APPLICATION CLAIMS THE BENEFIT OF PRIORIETY TO THE U. S. PROVISIONAL PATENT APPLICATION No. 60/233,739 FILED ON Sep. 19, 2000.

### TECHNICAL FIELD

The present invention relates generally to the field of obtaining preparations of antigens or preparations of antibodies that are particularly well suited for the detection of antigens or antibodies that relate to human etiological agents or human disease states or conditions. The preparations of antigens or preparations of antibodies are particularly useful for diagnosis, prognosis or treatment of infections with human etiological agents or human disease states or conditions.

### BACKGROUND

With the advent of genetic engineering methods the field of diagnostics has increasingly relied upon the identification of discrete antigens or groups of discrete antigens to make diagnostic and prognostic tests including immunoassays, such as test strips, in particular immunochromatographic test strips. One driving force in this direction has been the promise of inexpensive, abundant and efficacious diagnostic reagents. The present invention recognizes that this focused approach to biology does not efficiently mirror the course of infection with etiological agents or disease states or conditions. Rather, the present invention moves away from recombinant methodologies to utilize biology as it occurs to provide diagnostic, prognostic and vaccine compositions, particularly for human etiological agents and human disease states and conditions, that are more closely related to the natural biology of infection by etiological agents and disease states or condition.

### SUMMARY

The present invention recognizes that human antibodies can be utilized to identify and produce antigen preparations that are particularly useful for the detection, diagnosis or prognosis of human etiological agents or human disease states or conditions. The present invention provides a variety of aspects, including antigenic preparations, antibody preparations, methods of making such preparations and methods of using such preparations.

A first aspect of the present invention is a method for preparing a composition relating to a human etiological agent that includes: providing at least one preparation of antibodies comprising human antibodies optionally provided on a solid support; providing at least one preparation of at least one human etiological agent; contacting the preparation of at least one etiological agent with the preparation of antibodies; and isolating moieties bound to the preparation of antibodies to provide an isolated composition relating to a human etiological agent.

A second aspect of the present invention is a method for preparing a composition relating to a human disease state or condition, including: providing at least one preparation of antibodies comprising human antibodies; providing at least one preparation of at least one human disease state or condition; contacting the preparation of at least one human disease state or condition with said preparation of antibodies; and isolating moieties bound to said preparation of antibodies to provide an isolated composition relating to a human disease state or condition.

A third aspect of the present invention is a composition, preferably an antigenic composition, relating to a human etiological agent or a human disease state or condition made at least in part using at least one method of the present invention.

A fourth aspect of the present invention is a method of making an antibody preparation, including: providing a composition of the present invention, such as an antigenic composition; administering the composition to a subject; and obtaining a sample from the subject that includes antibodies. The present invention also includes a method of making a hybridoma or immortalized cell, including: providing a composition of the present invention, such as an antigenic composition; administering the composition to a subject; obtaining a sample from the subject that comprises antibody producing cells or their precursors; making a hybridoma or immortalized cell from the antibody producing cells or their precursors.

A fifth aspect of the present invention is an antibody, an antibody preparation, a hybridoma or immortalized cell, or an antibody made by such hybridoma or immortalized cell, using a method of the present invention.

A sixth aspect of the present invention is diagnostic, a test strip or a zone, such as a zone on a test strip that includes an antigenic composition or antibody composition of the present invention.

A seventh aspect of the present invention is a method for detecting an antibody that binds with an antigenic preparation relating to a human etiological agent, including: providing a sample from a subject; providing a composition of the present invention, such as an antigenic composition relating to an etiological agent; contacting the sample with the composition; and detecting the binding of one or more components of the sample with the composition.

An eighth aspect of the present invention is a method for detecting an antibody that binds with an antigenic preparation relating to a human disease state or condition, including: providing a sample from a subject; providing a composition of the present invention, such as an antigenic composition relating to a disease state or condition; contacting the sample with the composition; and detecting the binding of one or more components of the sample with the composition.

A ninth aspect of the present invention is a method for detecting an antigen that binds with an antibody preparation relating to a human etiological agent, including: providing a sample; providing a composition of the present invention, such as an antibody preparation of the present invention relating to an etiological agent; contacting the sample with the composition; and detecting the binding of one or more components of the sample with the composition.

A tenth aspect of the present invention is a method for detecting an antigen that binds with an antibody preparation relating to a human disease state or condition, including: providing a sample from a subject; providing a composition of the present invention, such as an antibody of the present invention relating to a disease state or condition; contacting the sample with the composition; and detecting the binding of at least one component of the sample with the composition.

### DETAILED DESCRIPTION OF THE INVENTION

### DEFINITIONS

Unless defined otherwise, all technical and scientific terms used herein have the same meaning as commonly understood by one of ordinary skill in the art to which this invention belongs. Generally, the nomenclature used herein and the manufacture or laboratory procedures described below are well known and commonly employed in the art. Conventional methods are used for these procedures, such as those provided in the art and various general references. Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Etiological agent” refers to any etiological agent, such as a bacterium, virus, parasite, fungus or prion that can infect a subject. An etiological agent can cause symptoms or a disease state, such as ranging from general malaise to moderate clinical symptoms such as retinitis, to more severe clinical symptoms or outcomes such as gastrointestinal distress, to even more sever symptoms or outcomes such as encephalitis, ulcers or even death. On the other hand, an etiological agent may not cause any appreciable clinical symptoms. A human etiological agent is an etiological agent that can infect a human subject. Such human etiological agents may be specific for humans, such as a specific human etiological agent, or may infect a variety of species, such as a promiscuous human etiological agent.

“Subject” refers to any organism, such as an animal or a human. An animal can include any animal, such as a companion animal such as a dog or cat, an agricultural animal such as a pig or a cow, or a pleasure animal such as a horse.

“Relating to an etiological agent” refers to a composition that is molecularly or physiologically, directly or indirectly, related to an etiological agent. For example, relating to an etiological agent includes moieties specifically or not specifically associated with the etiological agent, such as proteins, carbohydrates, lipids, fats, fatty acids, nucleic acid molecules, organelles and subsets or combinations thereof. Certain etiological agents produce toxins, such as enterotoxins, endotoxins or exotoxins. Examples of endotoxins include those produced by bacteria such as gram negative bacteria, such as Lipopolysaccharides (LPS) by Salmonellae or pyrogens. Examples of exotoxins are those produced by the Clostridia, such as botulism toxin produced by C. botulinum. Other toxins include those produced by C. tetani, C. perfringens, C. speticum, C. novyi, C. diptheriae, S. aureus, Y. pestis, B. pertussis, S. dysenteriae, V. cholerae and E. coli. Relating to an etiological agent also includes physiological responses to the etiological agent, such as but not limited to responses including inflammation, cytokine profiles, hematopoietic cell activation or humoral cell activation such as T-cell action or B-cell activation, encapsulation or the formation of cysts or the like.

“Preparation of an etiological agent” refers to a preparation that includes indicia (such as moieties such as but not limited to antigens or epitopes) of an etiological agent or a physiological response thereto. Preparations of an etiological agent preferably include antigens that relate to an etiological agent, including antigens derived from an etiological agent, antigens made by an etiological agent during the course of infection but are not part of the etiological agent itself, or antigens that are made by a subject in response to an etiological agent.

“Antibody” or “antibodies” refers to antibodies of any class or subclass or fragments, such as active fragments or any combination thereof. Active fragments of antibodies preferably include the Fv region of an antibody. Active fragments of antibodies can be made using methods known in the art, such as proteolytic digestion of samples including antibodies.

“Preparation of antibodies” refers to a sample that includes antibodies. Such samples can be crude, such a whole blood or serum or plasma, or can be partially purified, such as by crude separation methods such as molecular weight purification or ammonium sulfate precipitation, or can be substantially purified, such as by affinity chromatography for a class of antibody, subclass of antibody, or by binding with a particular antigen or epitope. Methods for such purification are known in the art, such as provided by Harlow and Lane, Antibodies, A Laboratory Manual, Cold Spring Harbor Press (1988).

“Human antibodies” refers to antibodies or preparations of antibodies that include antibodies derived at least in part from a human subject.

“Solid support” refers to any solid support, such as solid supports that are appropriate for immobilizing antibodies or antigens. A solid support is preferably in an appropriate format, such as a well, sheet, strip, bead or particle. Appropriate solid supports are known in the art and can be charged, neutral, magnetic or a combination thereof.

“Pooled antibodies” refers to a preparation of antibodies derived from at least two subjects. Pooled antibodies can be derived from members of the same species or different species, but are preferably derived from the same species. In one aspect of the present invention, pooled antibodies can be derived from the same subject over the course of time.

“Currently infected” refers to a subject that has been diagnosed as being infected with an etiological agent.

“Suspected of being infected” refers to a subject that has not been diagnosed as being infected with an etiological agent, but based on symptoms or other indicia, is more likely that not infected with an etiological agent.

“Exhibiting symptoms” of infection refers to a subject that exhibits at least one symptom of infection of an etiological agent and is less likely than more likely to be infected with an etiological agent. Subjects exhibiting symptoms of infection by an etiological agent are particularly directed to subjects that exhibit at least one symptom of an etiological agent that is particularly difficult to diagnose based on the particular etiology or pathology of an etiological agent, or the difficulty in diagnosis of infection by an etiological agent due to poor diagnostic potential of the etiological agent, such as for prion and certain viruses that are particularly difficult to cultivate or that biopsy samples are not particularly appropriate.

“Previously infected,” “suspected of previously being infected” and “previously exhibiting symptoms” of infection refers to a subject that previously, but no longer, meets the criteria of being currently infected, suspected of being infected or exhibiting symptoms of infection by an etiological agent. The subject may, however, still be infected by the etiological agent.

“Immobilized” refers to a moiety being irreversibly immobilized, reversibly immobilized, directly immobilized or indirectly immobilized on another moiety, such as a substrate, such as a solid support.

“Irreversibly immobilized” refers to the covalent linking of a moiety to another moiety, such as a substrate such as a solid support.

“Reversibly immobilized” refers to the non-covalent linking of a moiety to another moiety, such as a substrate such as a solid support.

“Directly immobilized” refers to one moiety being bound to another moiety, such as a substrate such as a solid support, without the use of another moiety. Direct immobilization includes covalent linking, passive absorption such as by short-range interactions, or specific absorption such as specific receptor ligand interactions, of one moiety to another.

“Indirectly immobilized” refers to one moiety being bound to another moiety, such as a substrate such as a solid support, with the use of another moiety. For example, a linker may be used to indirectly immobilize a moiety with a substrate. In the alternative, specific binding pairs such as antibody-antigen, receptor-ligand or avidin-biotin pairs can be used to indirectly immobilize a moiety on a substrate. For example, a substrate can be coated with one member of such a pair, such as avidin which, can bind with an antigen or antibody that is linked to the other member of the pair, such as biotin. In the alternative, a substrate such as a solid support can be coated to promote binding of a moiety to the substrate. Preferred coatings include polymers, particularly charged polymers, particularly positively charged polymers.

“Whole cells” refers to a preparation that includes a greater proportion of whole cells as opposed to portions of cells. The cells can be viable, non-viable or a combination thereof, wherein viable refers to the ability of the etiological agent to initiate an infection of a subject.

“Whole fungi” refers to a preparation that includes a greater proportion of whole fungi as opposed to portions of cells. The fungi can be viable, non-viable or a combination thereof, wherein viable refers to the ability of the etiological agent to initiate an infection of a subject.

“Whole viruses” refers to a preparation that includes a greater proportion of whole viruses as opposed to portions of viruses. The viruses can be viable, non-viable or a combination thereof, wherein viable refers to the ability of the etiological agent to initiate an infection of a subject.

“Whole parasites” refers to a preparation that includes a greater proportion of whole parasites as opposed to portions of parasites. The parasites can be viable, non-viable or a combination thereof, wherein viable refers to the ability of the etiological agent to initiate an infection of a subject.

“Whole prions” refers to a preparation that includes a greater proportion of whole prions as opposed to portions of prions. The prions can be viable, non-viable or a combination thereof, wherein viable refers to the ability of the etiological agent to initiate an infection of a subject.

“Cell free” refers to a preparation that includes a greater proportion of portions of cells as opposed to whole cells.

“Fungi free” refers to a preparation that includes a greater proportion of portions of fungi as opposed to whole fungi.

“Virus free” refers to a preparation that includes a greater proportion of portions of viruses as opposed to whole viruses.

“Parasite free” refers to a preparation that includes a greater proportion of portions of parasites as opposed to whole parasites.

“Prion free” refers to a preparation that includes a greater proportion of portions of prions as opposed to whole viruses.

“Crude preparation” refers to an unaltered sample that is obtained in vitro, ex vivo or in vivo.

“Partially purified” refers to a preparation that has been subjected to purification by nonspecific procedures, such as separation my molecular weight, charge, size, shape or other nonspecific physical property.

“Substantially purified” refers to a preparation that has been subjected to purification by specific procedures, such as separation by specific binding reactions, such as, for example, immunoaffinity or receptor ligand methods.

“In vitro” refers to procedures that take place outside of an organism or outside or organ culture. For example, in vitro cultivation procedures utilize appropriate growth media in the absence of viable tissues or organs. For example, bacteria can be in certain instances be cultivated in growth media and viruses can be cultivated in tissue culture using cell lines.

“Ex vivo” refers to procedures that take place outside of an organism that are not in vitro, such as in organ cultures or tissue cultures using samples from a subject, such as a volunteer or a cadaver. For example, certain etiological agents preferably are cultivated in organ samples or tissue samples.

“In vivo” refers to procedures that take place within an organism or embryo thereof. For example, certain etiological agents are preferably cultivated in whole organism, such as laboratory animals, or embryos thereof; such as in eggs such as avian eggs.

“Human disease state or condition” refers to a pathological condition, whether exhibiting symptoms or not, may or may not be directly or indirectly caused by an etiological agent. Preferably, a human disease state or condition includes disease state or conditions that are not directly caused by an etiological agent.

“Relating to a disease state or condition” refers to a composition that is molecularly or physiologically, directly or indirectly, related to a disease state or condition. For example, relating to a disease state or condition includes moieties specifically or not specifically associated with the disease state or condition, such as proteins, carbohydrates, lipids, fats, fatty acids, nucleic acid molecules, organelles and subsets or combinations thereof. Certain disease states or conditions result in the production of moieties that are associated with that disease state or condition, such as cell surface antigens or antigens that are shed into the host, such as prostate specific antigens or PSA. Relating to a disease state or condition also includes physiological responses to the disease state or condition, such as but not limited to responses including inflammation, cytokine profiles, hematopoietic cell activation or humoral cell activation such as T-cell action or B-cell activation, encapsulation or the formation of cysts or the like.

“Preparation of at least one disease state or condition” refers to a preparation that includes indicia (such as moieties such as but not limited to antigens or epitopes) of disease state or condition or a physiological response thereto. Preparations of a disease state or condition preferably include antigens that relate to a disease state or condition, including antigens derived from a disease state or condition, antigens made by a disease state or condition during the course of the disease state or condition, or antigens that are made by a subject in response to a disease state or condition.

“Currently having symptoms” of a disease state or condition refers to a subject that has been diagnosed as having a disease state or condition.

“Suspected of having symptoms” of a disease state or condition refers to a subject that has not been diagnosed as having a disease state or condition, but based on symptoms or other indicia, is more likely than not top currently have the disease state or condition.

“Exhibiting symptoms” of a disease state or condition refers to a subject that exhibits at least one symptom of a disease state or condition and is less likely than more likely to currently have the disease state or condition. Subjects exhibiting symptoms of a disease state or condition are particularly directed to subjects that exhibit at least one symptom of a disease state or condition that is particularly difficult to diagnose based on the particular etiology or pathology of a disease state or condition, or the difficulty in diagnosis of a disease state or condition due to poor diagnostic potential or that biopsy samples are not particularly appropriate.

“Previously having symptoms,” “previously suspected of having symptoms” and “having exhibiting symptoms” of a disease state or condition refers to a subject that previously, but no longer, meets the criteria set forth above for currently having symptoms, suspected of having symptoms and exhibiting symptoms of a disease state or condition.

“Relating to the structure or function of at least one tissue or organ” refers to the pathological effect of a disease state or condition. Various disease states or conditions tend to have structural or functional effects on particular tissues or organs. For example, cancers tend to have profound effects on the structure or function of the parent tissue, as do neurological disorders on the neurological system.

“Derived from the endoderm, ectoderm or mesoderm” refers particularly to proliferative disorders that derive from tissues or organs that were themselves derived from the embryonic endoderm, ectoderm or mesoderm.

“Cellular proliferative disorder” refers to disease state or conditions that are characterized by inappropriate proliferation of cells, tissues or organs.

“Cellular non-proliferative disorder” refers to disease state or conditions that are characterized by inappropriate non-proliferation or death of cells, tissues or organs.

“Cancer” refers to any of a variety of types of malignant neoplasms, which may metastasize to one or several sites, including carcinomas and sarcomas. Cancer can be associated with essentially all tissues or organs of the body, including particularly the skin, head, neck, thyroid, lung, liver, esophagus, stomach, pancreas, colon, rectum, anus, breast, cervix, endometrium, ovary, testis, prostate, kidney, bladder, central nervous system, soft tissue, bone and blood cells such as lymphocytes.

“Carcinoma” refers to a variety of malignant neoplasms derived from the epithelial tissues, occurring preferably but not exclusively in the skin and large intestine of both sexes, the bronchi, stomach and prostate in men, and the breast and cervix in women. Examples of carcinomas include, but are not limited to, acinar, acinic, acinoise, adenoid, adenoid squamous, adnexal, adrenal cortical, alveolar, basal cell, basal squamous, basisquamous, basaloid, basisquamous, basosquamous, breast, bronchiolar, bronchiolo-alveolar, bronchogenic, cervical, clear cell, colloid, cutaneum, cylindromatous, cystic, duct, ductal, embryonal, epidermoid, follicular, giant cell, thyroid, glandular, har-matrix, hepatocellular Hurthle, intermediate, intraductal, intraepidermal, inttraepithelial, invasive, kangri bum, large cell, latent, lateral aberrant thyroid, lenticulare, leptomeningeal, liver cell, lobular, lucke, medullary, melanotic, meningeal, mesometanephirc, metastatic, metatypical, microinvasive, mucinous, muceopidermoid, myxomatodes, noninfiltrating lobular, oat cell, papillary, primary, renal, sarchomatoid, scirrhous, secondary, signet ring cell, simplex, small cell, spiculated, spindle cell, squamous cell, transitional cell, V-2, verrucous, villous, Walker and wolffian duct.

“Sarcoma” refers to a connective tissue neoplasm, usually highly malignant, formed by proliferation of mesodermal cells. Examples of sarcomas include, but are not limited to alveolar soft part, ameloblastic, angiolithic, botryoid, deciduocellular, endometrial stromal, Eqing's , fascicular, giant cell, immunoblastics, Jensen's , juxtacoritcal osteogenic, Kaposi's , leukocytic, lymphatic, medullary, multiple idiopathic hemorrhagic, myelogenic, osteogenic, reticulum cell, rod cell, spindle cells and synoval.

“Adenocarcinoma” refers to glandular cancer or carcinomas, such as a malignant neoplasm of epithelial cells in glandular or gland-like patterns. Examples of adenocarcimomas include, but are not limited to acinic cell, bronchiolar, clear cell, Lucke's , mesonephric, mucoid, papillary and renal.

“Neoplasm” refers to an abnormal tissue that grows by cellular proliferation more rapidly than normal and can continue to grow after the stimuli that induced the new growth have been withdrawn. Neoplasm tends to show partial or complete lack of structural organization and functional coordination with the normal tissue, and tend to form a distinct mass of tissue that may be benign or malignant.

“Lymphoma” refers to a general term for ordinarily malignant neoplasms of lymph and reticuloendothelial tissues that tend to be present as apparently circumscribed solid tumors that include cells that appear primitive or resemble lymphocytes, plasma cells or histiocytes. Lymphomas tend to appear most frequently in lymph nodes, spleen or other normal sites of lymphoreticular cells. When disseminated, may invade the peripheral blood and manifest as leukemias. Lymphomas tend to be classified by cell type, degree of differentiation, and nodular or diffuse patterns. Examples of lymphomas include, but are not limited to Hodgkin's disease, Burkitts, follicular, histocytic, immunoblastic, Lenert's , Mediterranean, nodular, poorly differentiated lymphocytic (PDLL) and well-differentiated lymphocytic (WDLL).

“Benign” refers to the non-malignant characteristics of a neoplasm.

“Malignant” in the context of a neoplasm, refers to having the property of locally invasive and destructive growth and metastasis.

“Growth” refers to a localized proliferation of cells, which may be benign or malignant.

“Tumor” refers to a growth, particularly neoplasms.

“Neurodegenerative disease state or condition” refers to a disease state or condition that results in decreased function of the neurological systems, particularly the central nervous system, such as but not limited to Alzheimer's disease and Parkinson's disease

“Autoimmune disease state or condition” refers to a disease state or condition that results in the degradation of cells, tissues or organs of a subject due to the inappropriate action of the subject's immune system, such as, but not limited to, arthritis and lupus.

“Ischemic disease state or condition” refers to a disease state or condition that results in the degradation of cells, tissues or organs of a subject due to the lack of delivery or exchange of gasses to a location, such as, but not limited to, heart attack and stroke.

“Trauma disease state or condition” refers to a disease state or condition that results in the degradation of cells, tissues or organs of a subject to a trauma event, such as, but not limited to, blunt object injury or piercing injury.

“Therapeutic composition” refers to a compound, composition, article of manufacture or methods of making or using same that are useful to treat or aid in treating a subject for an indication, such as infection with an etiological agent or a disease state or condition.

“Diagnostic composition” refers to a compound, composition, article of manufacture or methods of making or using same that are useful to diagnose or aid in diagnosing a subject for an indication, such as infection with an etiological agent or a disease state or condition.

“Prognostic composition” refers to a compound, composition, article of manufacture or methods of making or using same that are useful to prognoses or aid in prognosing a subject for an indication, such as infection with an etiological agent or a disease state or condition.

“Administering” refers to providing a subject with a composition, such as a therapeutic composition, diagnostic composition or prognostic composition by an appropriate route of administration, at an appropriate dose using an appropriate regime with the purpose of an intended result, such as treating, diagnosing or prognosing infection with an etiological agent or a disease state or condition.

“Antibody producing cells” refers to cells, such as hematopoietic cells, such as B-cells that are producing, will produce or have produced antibodies.

“Precursors of antibody producing cells” refers to cells that can mature into antibody producing cells, such as stem cells for B-cells.

“Hybridoma” refers to a fusion of two cell types to result in a third cell, which is preferably immortalized. Preferably, hybridoma cells are made using antibody producing cells or precursors thereof such that the hybridoma produces monoclonal antibodies.

“Immortalized” refers to a cell whose life span or replicate potential has been extended by immortalization procedures. Immortalization procedures include the formation of hybridomas using an immortalized cell as one of the fusing cells, or by infecting a cell with a vector, such as a virus, that can impart immortalized characteristics on a cell. Such vectors preferably include transforming genes that can be selected based on the cell type to be immortalized.

“Test strip” refers to an article of manufacture or composition that includes one or more zones, such as, for example, one or more of the following in any appropriate configurations: sample application zone, reagent zone, detection zone and control zone. A test strip can be used to detect the presence or absence of an analyte, such as a chemical, antigen or antibody. Test strips are known in the art, particularly immunochromatographic and “dip” type test strips that are used to detect, for example, reproductive hormones, drugs of abuse, etiological agents or chemicals in samples, such as but not limited to blood or urine.

“Zone” such as a zone on a test strip refers to a locus on a test strip. A zone preferably includes a reagent, such as a chemical, antibody or antigen that is directly, indirectly, reversibly or irreversibly immobilized at such locus.

Other technical terms used herein have their ordinary meaning in the art that they are used, as exemplified by a variety of technical dictionaries.

### Introduction

The present invention recognizes that human antibodies can be utilized to identify and produce antigen preparations that are particularly useful for the detection, diagnosis or prognosis of human etiological agents or human disease states or conditions. The present invention provides a variety of aspects, including antigenic preparations, antibody preparations, methods of making such preparations and methods of using such preparations.

As a non-limiting introduction to the breath of the present invention, the present invention includes several general and useful aspects, including:

1) a method for preparing a composition relating to a human etiological agent;

2) a method for preparing a composition relating to a human disease state or condition;

3) a composition, preferably an antigenic composition, relating to a human etiological agent or a human disease state or condition made at least in part using at least one method of the present invention;

4) a method of making an antibody preparation using a composition of the present invention and a method of making a hybridoma or immortalized cell using a composition of the present invention;

5) an antibody, an antibody preparation, a hybridoma or immortalized cell, or an antibody made by such hybridoma or immortalized cell, using a method of the present invention;

6) a diagnostic, prognostic, test strip or zone, that includes an antigenic composition or antibody composition of the present invention;

7) a method for detecting an antibody that binds with an antigenic preparation relating to a human etiological agent;

8) a method for detecting an antibody that binds with an antigenic preparation relating to a human disease state or condition;

9) a method for detecting an antigen that binds with an antibody preparation relating to a human etiological agent; and

10) a method for detecting an antigen that binds with an antibody preparation relating to a human disease state or condition.

These aspects of the invention, as well as others described herein, can be achieved by using the methods, articles of manufacture and compositions of matter described herein. To gain a full appreciation of the scope of the present invention, it will be further recognized that various aspects of the present invention can be combined to make desirable embodiments of the invention.

I. Methods for Preparing Compositions Relating to Human Etiological Agents

The present invention provides a method for preparing a composition relating to a human etiological agent. One aspect of the present invention is a method for preparing a composition relating to a human etiological agent that includes: providing at least one preparation of antibodies comprising human antibodies optionally provided on a solid support; providing at least one preparation of at least one human etiological agent; contacting the preparation of at least one etiological agent with the preparation of antibodies; and isolating moieties bound to the preparation of antibodies to provide an isolated composition relating to a human etiological agent.

### Solid Support

The solid support, when used, is preferably is made of any appropriate material and is in a configuration that is useful in immunoseparation of materials or moieties. For example, the solid support can be made of polymers, plastics, cellulose, magnetic materials, derivatives thereof or any combination thereof. The solid support can be provided in any appropriate configuration, such as a well, sheet, strip, bead or particle, or any combination thereof. The solid support may be provided loose, such as in a powder form, particulate form, sheet form or strip form, or be provided in a contained structure, such as a column or test strip housing. Preferred materials for a solid support include polystyrene, polypropylene, cyclo-olifins, cellulose, glass, nitrocellulose, lint and other appropriate materials as they are known in the art.

Importantly, the solid support is optional in the present invention. Antibody-antigen reactions can take place in solution from which the resultant complexes can be recovered, particularly when the relative concentrations of the antibody and antigen are such that a crosslinked precipitate forms.

### Preparation of Antibodies

Antibodies for use in the present invention can be derived from any appropriate source, such as a subject, such as a single human at a point in time. Antibody preparations can also be pooled antibody preparations from a single subject or a plurality of subjects and can be made by collecting serum or plasma from one or more subjects or by purchasing pooled serum or plasma samples, such as through New York Biologics, Inc. (Southampton, N.Y.). Antibody preparations can be made from blood or serum or plasma samples using methods known in the art to make partially purified or substantially purified preparations. Preferably, antibody preparations are made using ammonium sulfate precipitation of serum or plasma followed by dialysis, ion exchange such as ion exchange chromatography or by affinity methods such as affinity chromatography, preferably immunoaffinity chromatography. Antibody preparations can be of any class of subclass or antibodies, or any combination thereof, such as the classes IgG, IgM, IgE, IgA or IgD. The desired class of antibodies can be obtained in higher proportions depending on the physiological site from which a sample is obtained and the time during the course of infection that the sample is taken.

A combination of methods can be used to make a preparation of antibodies useful in the present invention. For example, a preparation of antibodies can be made using a first method, such as non-selective, semi-selective or selective method, such as for example, precipitation, filtration or affinity methods. This first method can be considered an enrichment method. Precipitation methods can be accomplished using antigens or precipitating agents, such as ammonium sulfate. Filtration methods include a variety of methods known in the art that result in the separation of moieties based on their size, shape or charge, such as size exclusion filtration or chromatography, size filtration through a filter, ultrafiltration, PAGE, SDS-PAGE, isoelectric focusing or the like. Affinity methods include methods that separate moieties based on their affinity for a receptor or ligand, such as affinity chromatography, such as immunoaffinity chromatography or receptor or ligand chromatography. Preferably, this first method is nonselective or semi-selective in nature.

This first preparation of antibodies can be enriched using a second method, such as nonselective, semi-selective or selective methods, such as precipitation methods, filtration methods or affinity methods. This second method can be considered an enrichment method. Preferably, the first method is semi-selective or non-selective, such as filtration methods or precipitation methods and the second method is selective, such as affinity methods. This need not be the case, however, and the various steps can be intermingled and more than one method can be used to make a preparation of antibodies. Preparations of antibodies using one or more of such methods result in enriched antibodies.

The antibodies present in these preparations, such as a first enriched preparation or a second enriched preparation can be concentrated. Appropriate methods include, but are not limited to, ultrafiltration or lyophilization.

Antibody preparations can be made using at least in part serum or plasma samples from a variety of subjects. Preferably, antibody preparations are made using serum or plasma samples from at least one subject currently infected with, suspected of being infected with, or exhibiting symptoms of, infection with a human etiological agent. For the present invention, a subject need not be human, but is preferably human. Alternatively, antibody preparations can be made using at least in part serum or plasma samples from at least one subject previously infected with, previously suspected of being infected with, or previously exhibiting symptoms of, infection with a human etiological agent.

Etiological agents of the present invention can be any etiological agents, preferably etiological agents that infect only humans, but also promiscuous human etiological agents. Where the etiological agent is a promiscuous human etiological agent, an appropriate animal model, such as a laboratory or farm animal, can be used as a source of antibodies.

### Solid Support with Preparation of Antibodies

Antibodies can be immobilized upon such solid supports using a variety of methods and reagents known in the art to irreversibly immobilize, reversibly immobilize, directly immobilize or indirectly immobilize antibodies. For example, antibodies can be directly immobilized on a solid support by passive absorption, selective absorption such as a solid support bound with avidin binding an antibody linked to biotin (or vis-a-vis) or can be covalently linked to a solid support. Alternatively, antibodies can be indirectly linked to a solid support using covalent linkers or by using binding pairs, such as solid supports having bound thereto antibodies, which bind the antibodies of interest, preferably, the Fc regions. Alternatively, the solid support can have bound thereto a reagent, such as avidin or biotin, that binds with an antibody respectively linked to biotin or avidin.

### Etiological Agent

Etiological agents of the present invention can be any etiological agents, preferably etiological agents that infect only humans, but also promiscuous human etiological agents. Etiological agents can be, for example, bacteria, viruses, fungi, parasites and prions.

### Bacteria and Fungi

Bacteria are unicellular prokaryotic organisms and are the smallest organisms that utilize foodstuffs for growth and self-replication. They can be rod shaped, cocci spiral or filamentous and are about 0.2 to about 2 micrometers in diameter. Bacteria possess a cell wall that can be used to identify them as Gram positive or Gram negative. Gram positive cells consist of a cytoplasmic membrane and a thick peptidoglycan layer with an optional variable outer layer referred to as a capsule. Gram negative cells consist of a cytoplasmic or inner membrane surrounded by a thin peptidoglycan layer to which an outer membrane is anchored and can have an outermost variable capsule. Both Gram negative and Gram positive bacterial can be etiological agents and human etiological agents. A capsule is an extracellular polymer than can contribute to the pathogenicity of bacteria by contributing to the invasiveness of pathogenic bacteria by, for example, protecting the organism from a host's defense system such as by phagocytosis.

Pathogenic bacteria cause disease in humans by a variety of different methods and mechanism, such as by an invasive mechanism that can cause damage to the host cells, production of toxins that may be transported throughout the subject, and can effect a hypersensitive response. Invasion of bacteria within a subject such as a human can encompass surface colonization of the subject, such as on damaged skin, in the lungs, on cells or tissues and penetration and damage of cells or tissues. Pathogenic bacteria that can survive and multiply in phagocytotic cells can cause more chronic diseases such as tuberculosis. Bacteria that are subject to phagocytosis can damage tissue only so long as they are outside of the phagocytotic cells and therefore usually cause acute symptoms for short duration. Production of antibodies that complex with the invading entity renders it susceptible to phagocytosis.

There are numerous diseases in humans caused by pathogenic bacteria that can be effected by the present invention including Helicobacter pylori, a Gram-negative bacterium that can colonize the mucosal lining of the stomach and proximal duodenum, that is the causative agent of chronic gastritis and peptic ulcers, primary gastric B-cell lymphoma, and in the long term, increases risk to gastric adenocarcinoma and may be associated with short stature in young girls.

Other bacteria that cause human diseases include, but are not limited to, those of the genus Bacillus and can cause diseases such as meningitis, endocarditis, endophtalmitis, conjunctivitis, and acute gastroenteritis. Staphylococcus, such as S. aureus, can cause different infections in humans such as pyrogenic infections, toxic shock syndrome, pneumonia, meningitis, emphysema, endocarditis, or sepsis. S. saprophyticus can cause urinary tract infection in young women. Streptococcus, such as S. pnemoniae, can cause pneumonia, sinusitis, otitis, bronchitis, bacteremia, and meningitis and Klebsiella, such as K. pneumoniae is another respiratory pathogen. Examples of endotoxin producing bacteria that cause diseases in humans include Clostridum tetani that can cause tetanus, Corynebacterium diphtheria causes diphtheria, Bordetalla pertussis causes whooping cough, and Shigella dysenteriae causes dysentery.

Human disease causing bacteria from the genus Rickettsia include R. prowazcki and R. typhi that cause typhus, R. rickettsi that can cause Rocky Mountain spotted fever, and R. akari that causes rickettsial pox. Legionella phneumophila and L. micadadei, to a lesser extend, cause diseases in humans such as pneumonia. The prokaryotic genus Bacteroides contains species, such as B. fragilis, that have capsules to which infected humans develop antibodies, and can cause bacteremia and be involved in causing peritonitis. Bacteria from the genus Mycobacterium, including M. tuberculosis and M. leprae, can cause chronic diseases and lesions in humans.

Species in the family Salmonellae that are transmitted from animal or animal products to humans can cause enteritis, systemic infection and enteric fever. Another groups of bacteria that normally have animal hosts but can cause disease in humans include Yersinia pesitis (plague), Y. pseudotuberculosis, Y. enterocolitica, Franscisella tularensis, and several species of Pasturella. These examples reflect only a small portion of a much larger population of bacterial human pathogens that can be utilized by the present invention. Generally, a wide variety of bacterial pathogens that can cause infections in human subjects can be from a variety of genus or groups of bacteria, including, for example, Corynebacteria, Pneumococci, Strrptococci, Staphylococci, Neisseriae, The Enteric Bacilli, Bacteroides, Pseudomonas and other Nonfermential Bacilli, Yersinia, Franscisella, Pasteurella, Brucella, Hemophilus, Bordetella, the Aerobic Spore-Forming Bacilli, The Anaerobic Spore-Formic Bacilli, Clostridia, Mycobacteria, Actinomycetes, Spirochtes, Richettsiae, Chlamydiae and Mycoplasmas.

### Viruses

Another groups of etiological agents that can be effected by the present invention are viruses. Viruses are generally divided into three groups: animal viruses, plant viruses and bacterial viruses. The present invention is particularly directed towards animal viruses, such as those that can specifically infect humans or can promiscuously infect humans and other animals.

Viral particles include a genome of DNA or RNA, in single stranded or double stranded form, in a linear or circular form, in a single molecule or multiple segments, or combinations thereof. The genome can be enclosed in a capsid, protein coat or a lipid or lipid-protein layer which protects the genome and aids in attachment and penetration of the virus or a portion thereof into a host cell. Some viruses present a naked capsid to the environment such as an immune system, such as picoma viruses, whereas other viruses present envelopes that can include proteins to the environment, such as with retroviruses.

Viruses depend on the mechanism of the host cell to replicate. Maturation of viruses consists of synthesis of the nucleic acid and protein or proteins of the capsid, if present, and other viral specific proteins, assembly into mature viral particles and release from the host cell by a variety of mechanisms, such as rupture of the host cell or budding. Viral DNA, or DNA produced from a viral genome such as RNA, can become incorporated into the genome of the host cell and can become dormant or be active. Alternatively, the virus in a cell to replicate and escape or shed from the host cell to infect other cells can utilize the cellular function of the host cell. The host cell's cellular function can be utilized by the viral genome and ancillary proteins in order to effect viral replication, such as through the production of a variety of viral proteins, some of which become incorporated into a mature virus and others that do not. Some viral proteins can be expressed or presented on the surface of infected cells, such as in the case have enveloped viruses, such as HIV.

Upon infection or invasion of a human by a specific virus, antibodies can be produced by the host that bind to viral proteins, such as capsid proteins, proteins in an envelope, or other proteins in a virus particle, on a virus particle or that are produced by a virus-infected cell. In addition, a cellular immune response can be mounted against viral infected cells that present foreign antigens on their cell surface.

Viruses of humans can enter the body and cells thereof by a variety of methods. For example, certain viruses enter orally or by way of major or minor trauma, such as Hepadnaviridae that can cause Hepatitis B.; Papillomavirus that can cause different types of diseases including warts and cervical cancer; Herpesvididae, which can cause Herpes simplex 1 and 2; and Poxviridae, which can cause molluscum contagiosum, cowpopx, orf, milker's nodes, and vaccinia. Examples of viruses that enter by arthropod bites include Alphavirus; Plaviviridae that can cause different diseases including ebola; Poxviridae that can cause tanapoxivurs; Orbivirus that can cause Colorado tick fever; and Bunyaviridae that can cause La Corse, sandfly fever, and Rift Valley fever. Two examples of viruses that can enter a human via bite from a vertebrate are Rhabdoviridae that can cause rabies, and Herpeviridae that can ca B virus. Examples of viruses that are contracted by way of the genital tract include Papillomaviruses, Herpesviridae that can cause Herpes simplex, and Retroviridae that can cause HTLV-III.

Generally, a wide variety of viral pathogens that can cause infections in human subjects can be from a variety of classifications, such as, for example Hepadnaviruses, Papovaviruses, Adenoviruses, Herpesviruses, Poxviruses, Paroviruses, Picornaviruses, Caliciviruses, Togaviruses, Flaviviruses, Orthomyxoviruses, Paramyxoviruses, Coronaviruses, Arenaviruses, Bunyaviruses, Rhabdoviruses, Filoviruses, and Reoviruses. See generally, Davis et al., Microbiology, 3^{rd} rd edition, Harper & Row, Philadelphia (1980) and White et al., Medical Virology, 3^{rd} rd edition, Academic Press, Orlando (1986).

### Parasites

Parasites are organisms that reside on or within a host in order to obtain nutrients for growth and reproduction. Parasites of humans may be pathogenic to its host by means of invasiveness, establishment and multiplication, and toxigenicity. Bacteria and viruses are parasites as well as other microbes, flukes and worms. The latter forms of parasites of humans can be divided into different categories such as; flagellates that consist of the intestinal and genitourinary flagellates including species of Giardia, Trichomonas, Retortamonas, Dientamoeba, Enteromonas, and Chilomastix, and the blood and tissue flagellates including Trypanosoma and Leishmania; ameboid, such as Entamoeba, Endolimax, Iodamoeba, Naegleria, and Acanthamoeba; those parasites to humans that have complex lifecycles usually involving two different hosts and include species of Plasmodium, Isopora, Toxoplama, and Babesia; the parasite Balatidium coli, a ciliated protozoan is parasitic to both humans and pigs; flatworms that include tapeworms and flukes; and the wormlike, unsegmented roundworms. Examples of some specific parasites that cause diseases to humans include Giardia lambelia that when found in large numbers in the intestine can cause acute or chronic diarrhea. Trichomonas vaginalis causes trichomoniasis in humans. Trypanosoma brucei rhodesiene and Trypanosoma brucei gambiense, transmitted by tsetse flies, cause African sleeping sickness and Trypanosoma cruzi causes Chagas' disease. Different species of Leishmania can cause leishmaniasis such as Oriental sore, espundia, and kala-azar. Entamoeba histolytica is a common parasite of the large intestine of humans, other primates and some other animals and can cause extreme abdominal tenderness, amoebic dysentery, and dehydration in humans. Species of Plasmodium that include P. vivax, P. ovale, P. malariae, P. falciparum, and P. knowles when transmitted to humans by the Anopheles mosquito can cause malaria. Cryptosporidium species, parasites of some rodents, fowl, and animals, can infect the human intestine and cause severe diarrhea. Pneumocystis carinii, common in animals in nature, can cause interstitial plasma cell pneumonitis in infants, the elderly, and AIDS patients. Ascariasis lumbricoides is a common roundworm that can inhabit the human intestine and the nematode Trichinella spiralis causes trichinosis in humans, Angiostongylus cantonensis can cause eosinophilic meningoencephalitis, Capillaria philippinensis causes capillariasis, and Wuchereria bacrofti and Brugis malayi can cause filariasis. The giant intestinal fluke Fasciola hepatica can cause fascioliasis, and different species of Schistosoma can cause schistosomiasis. These examples and numerous other human parasites can effect by the present invension.

### Fungi

Another groups of etiological agents that can be effected by the present invention are fungi. See generally, Davis et al., Microbiology, 3^{rd} edition, Harper & Row, Philadelphia (1980), Baron et al., Diagnostic Microbiology, 8^{th} Edition, The C.V. Masby Company, St. Louis (1990), O'Learly, CRC Practical Handbook of Microbiology, CRC Press, Boca Raton (1989) and Jawetz et al., Review of Medical Microbiology, 17^{th} Edition, Appleton & Lange, Norwalk (1987).

There are about 100 known species of yeast and molds that can cause disease in humans. They are grouped into three categories; superficial, subcutaneous, and deep or systemic. Superficial fungal infections affect the skin, hair and nails of an individual but do not invade deeper tissue and are not usually health threatening. Subcutaneous fungal infections must be introduced into the subcutaneous tissue from where lesions can spread. Deep fungal infections can be health threatening and, in a minor portion of a population of infected people, can be fatal. The deep infection can result in granuloma and necrosis and abscess formation.

Examples of disease causing fungi of the subcutaneous type include Sporothrix scheneckii that can cause sporotrichosis when introduced into the skin and can spread along lymphatics draining the area. Several species of black mold including Plialophora verrucoa, Plialophora pedrosoi, and Cladosporium carrionii can cause chromomycosis, a slowly progressive granulomatous infection of skin. Several fungi including Petriellidium boydii, and different species of Madurella, Phiialophora, and Acremonium can cause mycetoma, swollen lesions with granules that are compact colonies of the infecting agent. Petriellidium boydii can also cause opportunistic disease of lungs and other organs.

Deep or systemic fungal mycoses usually infect humans by inhalation and often are asymptomatic. Within this group is Coccidioides immitus, the arthrospores of which can be inhaled and cause a respiratory infection that can become symptomatic. Histoplasma capsulatum can cause histoplasmosis, an intracellular mycosis of the reticuloendothelial system, and a heavy infection may result in pneumonia. Blastomyces dermatitidis grows as a budding cell in infected individuals and can cause the chronic granulomatous disease blastomycosis. And the causative agent for paracoccidioidomycosis is the deep infection fungus Paracoccidiodes brasiliensis.

There are also opportunistic fungi that can cause disease in humans that have a predispose condition. An example of opportunistic organisms are Candida albicans and related yeasts, normal flora of the respiratory, gastrointestinal, and female genital tracts, that can produce systemic disease. Candida can also cause bloodstream invasion, thrombophlebtis, endocarditis, and other infections of other organs when introduce intravenously. And Cryptococcus neoforms can cause disease in humans that inhale a massive amount into their lungs, but can also be responsible for an opportunistic infection of cryptococcosis.

### Prions

Prions, protein infectious particles, are small proteins, less than 50 nanometers in diameter, that are thought to be responsible for several neurodegenerative diseases in mammals. They are thought to be an alternate form of a protein normally found in the brain, that has a different folded shape than the normal protein. When the infective agent is transmitted to a host the prions apparently have the ability to affect the shape of the normal proteins and thereby increase their number. In humans prions are the possible causative agent of Fatal Familial Insomnia and the spongiform encephalopathies Kuru and Creutzfeld-Jakob disease.

### Preparation of Etiological Agents

Preparations of etiological agents can be made using appropriate methods known in the art. For example, etiological agents can be cultured in vitro when appropriate and possible. Alternatively, etiological agents can be cultured in organ culture or tissue cultures ex vivo. Furthermore, etiological agents can be cultivated in vivo in subjects, such as appropriate animals or in human volunteers or humans that are infected with an etiological agent. When the preparation of an etiological agent is derived from an animal or human, preparations relating to the etiological agent may result.

For etiological agents grown in vitro, an etiological agent is contacted with an appropriate growth medium and under appropriate conditions, for example temperature and gasses, such that the etiological agent can propagate and form a crude preparation relating to an etiological agent.

For etiological agents grown ex vivo, an etiological agent is contacted with an appropriate organ culture or whole tissue culture, such as derived from an animal, human volunteer or human cadaver, and placed under appropriate culture conditions such that the etiological agent can propagate and form a crude preparation relating to an etiological agent.

For some etiological agents, in vivo samples from animals, human volunteers or human cadavers can be used to provide or form crude preparations relating to an etiological agent. Some etiological agents are not particularly suited for in vitro or ex vivo cultivation due to their fastidiousness or the nature of the etiological agent. For example, a preparation relating to an etiological agent can include a sample obtained from a subject, such as blood, urine, tissue or organ samples, such as biopsies. Different etiological agents, based on their pathology, can produce relatively large quantities of an etiological agent or portions thereof, such as antigens or whole or partial etiological agents. For example, gastrointestinal etiological agents tend to replicate to relatively high numbers, such as the case of bacteria, parasites or viruses.

Other etiological agents form partial etiological agents, such as in the case of hepatitis B virus, during the course of an infection. Still other etiological agents form relatively high amounts of antigens separate from the whole etiological agent itself, such as in the case of virus infection where antigens can form or be presented on the surface of infected cells or shed into the surrounding tissues, organs or fluids. Still other etiological agents cause a subject to mount a physiological response to an infection, such as necrosis, inflamation or immunological responses. Preparations can include moieties that are related to such physiological responses, such as antigens.

In one particularly preferred aspect of the present invention, a preparation relating to a human etiological agent includes antigens, including the whole etiological agent, portions of an etiological agent or antigens relating to a physiological response to the etiological agent. Preferably, the preparation includes antigens that are unique to the etiological agent, but that need not be the case. For example, antigens from one etiological agent may cross react with antigens from another etiological agent, particularly similar or analogous antigens, particularly when the etiological agents are taxonomically related, such as the relatively close taxonomical relationship between members of the genus Salmonella. Furthermore, different etiological agents can cause, induce or prompt the same or similar physiological response in a subject, which does not detract from the present invention because the physiological response can be correlated with other tests or clinical indicia of an etiological agent, such as unique symptoms.

Preferably, a preparation relating to an etiological agent is taken from a locus of a subject where there is a relatively high probability of obtaining indicia of the etiological agent, such as antigens. The skilled practitioner recognizes that different etiological agents have target locations in a subject, such as particular fluids, solids, tissues or organs, or that a physiological response is greater in a particular fluid, solid, tissue or organ of a subject. The choice of an appropriate sample from which to make a preparation relating to an etiological agent can thus be made based on the etiological agent in question. For example, infections with gastro-intestinal etiological agents such as bacteria, viruses and parasites, tend to result in the presence of antigens in blood, serum or plasma, stool, urine and in the tissues of the infected subject. In such cases, appropriate samples can be used to make preparations relating to etiological agents, including human etiological agents.

These crude preparations can include whole etiological agents, such as whole cells, whole viruses, whole parasites, whole fungi, whole prions or combinations thereof. Alternatively, these crude preparations can include a cell free preparation, a virus free preparation, a fungi free, a parasite free preparation or prion free preparation. The particular characteristics of the crude preparation can be determined by the particular culturing conditions, be they in vitro, ex vivo or in vivo, and can be dictated by the particular biology of the etiological agent.

Crude preparations can also include supernatants, culture media, or filtrates or washes from cultures in vitro. For example, a culture of etiological agents can be grown and the etiological agent mass separated from the growth media by an appropriate method, such as filtration or centrifugation, to provide a supernatant or filtrate preparation and an etiological agent mass preparation. The etiological agent mass preparation can be further washed with media or buffer of different characteristics to provide additional crude preparations. In one aspect of the present invention, such etiological agent mass preparations can be washed with solutions of differing ionic strength, polarity or other characteristic such that antigens on or within the etiological agent mass, such as etiological agents, are stripped away or separated from the etiological agent mass.

Crude preparations can also include supernatants, culture media, or filtrates or washes from cultures ex vivo or in vivo preparations. For example, ex vivo or in vivo preparations, such as organ cultures or tissue cultures, organs or tissues, fluids or other samples, can be washed, infiltrated, homogonized or otherwise broken up, treated with proteases or other agents to separate tissue, organ or cellular structures, in order to free, suspend or otherwise provide a crude preparation. These preparations can be filtered or centrifuged to separate debris from the fluid portion of the sample to form a solid portion and a fluid portion, each of which being crude preparations. The solid portion can be further washed with media or buffer of different characteristics to provide additional crude preparations. In one aspect of the present invention, the solid portion can be washed with solutions of differing ionic strength, polarity or other characteristic such that antigens on or within the solid portion, such as etiological agents or cellular debris, tissue debris, organ debris or the like are stripped away or separated from the solid portion. In another aspect of the present invention, an organ or tissue can be infiltrated with solutions of differing characteristics, such as the infiltrate is collected. For example, an organ or tissue can be infiltrated with solutions of differing ionic strengths or polarity and the infiltrate collected.

The crude preparations can be used as is, or can be further treated or processed. For example, a crude preparation can be irradiated to inactivate etiological agents. A crude preparation can also be treated or processed to lyse or disrupt cells, viruses, parasites or prion. For example, a French press can be utilized to disrupt etiological agents, as can detergents, enzymes, temperature, freeze-thaw and other biological, mechanical and chemical methods.

Crude preparations can also be further treated or processed to provide partially purified preparations or substantially purified preparations. For example, a crude preparation can be partially purified using non-specific methods of separation or purification, such as size exclusion chromatography, ion exchange chromatography, precipitation such as through selective or semi-selective precipitation such as ammonium sulfate precipitation, HPLC, FPLC, hydophobic chromatography, reverse phase chromatography, isoelectric focusing, electrophoresis, SDS-PAGE, non-denaturing PAGE and the like. Also, a crude preparation can be substantially purified using specific methods of separation or purification, such as, for example, affinity chromatography. In this aspect of the present invention, specific binding reactions, such as receptor ligand or antibody-antigen binding can be used to isolate particular moieties, such as antigens. Bound moieties can be recovered using methods known in the art, such as using salt or salt gradients, the use of chaotropic or antichaotropic agents, detergents or shifts in pH, particularly where affinity chromatography using a solid support, such as in a column, is used.

### Isolating

Once a preparation, such as antigens is bound to a solid support, such as by binding to an antibody or antibodies bound to a solid support, the bound preparation can be recovered using methods known in the art. For example, if a column of solid support having immobilized antibodies bound with antigens from a preparation, the column can be rinsed or washed to remove non-bound entities. Solutions of different ionic strength, or a salt gradient, can be used to wash the column to elute antigens bound to the antibodies immobilized on the solid support. Fractions eluted from the column can be collected and screened for desired activity, such as binding with antibody preparations that include, or are suspected of including, antibodies that bind with an etiological agent. Depending on the character of the solid support, additional separation methods can be used. For example, if the solid support is magnetic in character, then a magnetic field can be used to temporarily localize the magnetic solid support so that the solid support can be washed or rinsed such that unbound material can be removed from the preparation. In this way, a composition including moieties relating to an etiological agent, such as a human etiological agent, results. The composition can be stored in a variety of conditions, such as liquid, frozen, suspended or lyophilized. The particular method of storing the composition is one of convenience and can be related to the intended use and shelf-life of the composition, and the stability of the composition under a variety of conditions, such as temperature, humidity and freeze-thaw.

Screening of compositions of the present invention for binding with antibodies in an appropriate preparation can use, for example, labeled binding reagents to detect the binding of antigen to antibody through a variety of formats, including competitive assays, sandwich assay, direct assays or indirect assays. One preferred method includes Western Blotting. For example, a composition of the present invention can be immobilized upon a solid support. An antibody preparation from a subject, such as a human, known or suspected of containing antibodies relating to an etiological agent, can be contacted with the antibodies on the solid support. A secondary antibody preparation, such as alkaline phosphatase labeled mouse anti-human antibodies is contacted with the solid support. Binding of the labeled mouse anti-human antibodies to human antibodies bound to the solid support, such as bound to antigens bound to the solid support, can be detected using appropriate substrates for the enzyme, such as pro-chromogenic substrates. Conversion of the pro-chromogenic substrate can be converted to a chromogen by cataltyic entities bound to the antibody, indicating that the antigen bound with the antibody. Compositions of the present invention that bind with such antibody preparations are preferred compositions of the present invention and can be provided singly or in combination to form a composition of the present invention.

A variety of detectable labels are available, as are a variety of assay formats, and can be used in the present invention. For example, detectable labels include radioactive, chromogenic, fluorescent, luminescent, chemiluminescent and the like. Assay formats that are preferred include sandwich assays, direct assays and indirect assays as they are known in the art. The choice of a particular format can be made by the skilled artisan based on preferences of assay formats, labels, sensitivity and available reagents or kits.

If a solid support is not used in the present invention, antibodies bound with antigen in solution, preferably form a precipitate that includes antibodies cross-linked with antigen. The precipitate, a crude preparation, can be washed using appropriate buffers and environmental conditions, and solubilized. Antibodies can be separated from antigen using a variety of methods, such as affinity chromatography or size exclusion chromatography, for example. The resulting preparation can be characterized as partially purified or substantially purified, depending on the methods used and the purity of the resulting composition.

### Further Processing

The compositions made using methods of the present invention can be further purified using non-specific purification methods or specific purification methods. For example, nonspecific purification methods include, but are not limited to, ion exchange chromatography, size exclusion chromatography, electrophoresis, non-denaturing electrophoresis, denaturing electrophoresis, PAGE, SDS-PAGE, isoelectric focusing, blotting, selective precipitation and centrifugation. Specific purification methods include, but are not limited to, affinity chromatography and immunochromatography. These further processed compositions can be screened for desirable characteristics, such as binding with an appropriate antibody preparation, using screening methods discussed herein.

II. Methods for Preparing Compositions Relating to Human Disease State or Conditions

The present invention provides a method for preparing a composition relating to a human disease state or condition. One aspect of the present invention is a method for preparing a composition relating to a human disease state or condition, including: providing at least one preparation of antibodies comprising human antibodies; providing at least one preparation of at least one human disease state or condition; contacting the preparation of at least one human disease state or condition with said preparation of antibodies; and isolating moieties bound to said preparation of antibodies to provide an isolated composition relating to a human disease state or condition.

### Solid Support

The solid support, when used, is preferably is made of an appropriate material and is in a configuration that is useful in immunoseparation of materials or moieties. For example, the solid support can be made of polymers, plastics, cellulose, magnetic materials, derivatives thereof or any combination thereof. The solid support can be provided in any appropriate configuration, such as a well, sheet, strip, bead or particle, or any combination thereof. The solid support may be provided loose, such as in a powder form, particulate form, sheet form or strip form, or be provided in a contained structure, such as a column or test strip housing. Preferred materials for a solid support include polystyrene, polypropylene, cycloolefins, cellulose, glass, nitrocellulose, lint and other appropriate materials as they are known in the art.

Importantly, the solid support is optional in the present invention. Antibody-antigen reactions can take place in solution from which the resultant complexes be recovered. Particularly when the relative concentrations of the antibody and antigen are such that a cross-linked precipitate forms.

### Preparation of Antibodies

Antibodies for use in the present invention can be derived from any appropriate source, such as a subject, such as a single human at a point in time. Antibody preparations can also be pooled antibody preparations from a single subject or a plurality of subjects and can be made by collecting serum or plasma from one or more subjects or by purchasing pooled serum or plasma samples, such as through Sigma Chemical (St. Louis, Mo.). Antibody preparations can be made from blood or serum or plasma samples using methods known in the art to make partially purified or substantially purified preparations. Preferably, antibody preparations are made using ammonium sulfate precipitation of serum or plasma followed by dialysis, ion exchange chromatography or affinity chromatography. Antibody preparations can be of any class of subclass or antibodies, or any combination thereof, such as the classes IgG, IgM, IgE, IgA or IgD. The desired class of antibodies can be obtained in higher proportions depending on the physiological site that a sample is taken and the time during the course of a disease state or condition that the sample is taken.

Antibody preparations can be made using at least in part serum or plasma samples from a variety of subjects. Preferably, antibody preparations are made using serum or plasma samples from at least one subject currently having, suspected of having or exhibiting symptoms of a disease state or condition. For the present invention, a subject need not be human, but is preferably human. Alternatively, antibody preparations can be made using at least in part serum or plasma samples from at least one subject previously having, suspected of having or exhibiting symptoms of a disease state or condition.

A combination of methods can be used to make a preparation of antibodies useful in the present invention. For example, a preparation of antibodies can be made using a first method, such as non-selective, semi-selective or selective method, such as for example, precipitation, filtration or affinity methods. This first method can be considered an enrichment method. Precipitation methods can be accomplished using antigens or precipitating agents, such as ammonium sulfate. Filtration methods include a variety of methods known in the art that result in the separation of moieties based on their size, shape or charge, such as size exclusion filtration or chromatography, size filtration through a filter, ultrafiltration, PAGE, SDS-PAGE, isoelectric focusing or the like. Affinity methods include methods that separate moieties based on their affinity for a receptor or ligand, such as affinity chromatography, such as immunoaffinity chromatography or receptor or ligand chromatography. Preferably, this first method is nonselective or semi-selective in nature.

This first preparation of antibodies can be enriched using a second method, such as nonselective, semi-selective or selective methods, such as precipitation methods, filtration methods or affinity methods. This second method can be considered an enrichment method. Preferably, the first method is semi-selective or non-selective, such as filtration methods or precipitation methods and the second method is selective, such as affinity methods. This need not be the case, however, and the various steps can be intermingled and more than one method can be used to make a preparation of antibodies. Preparations of antibodies using one or more of such methods result in enriched antibodies.

The antibodies present in these preparations, such as a first enriched preparation or a second enriched preparation can be concentrated. Appropriate methods include, but are not limited to, ultrafiltration or lyophylization.

Disease states or conditions of the present invention can be any disease state or condition that only affect humans. Alternatively, the disease state or condition can affect an animal that has been modified or genetically engineered, other than by selective breeding, to be an appropriate animal model, or an animal that is an appropriate animal model for the disease state or condition, including animals that have been selectively breed.

### Solid Support with Preparation of Antibodies

Antibodies can be immobilized upon such solid supports using a variety of methods and reagents known in the art to irreversibly immobilize, reversibly immobilize, directly immobilize or indirectly immobilize antibodies. For example, antibodies can be directly immobilized on a solid support by passive absorption, selective absorption such as a solid support with bound avidin binding an antibody linked to biotin (or vis-a-vis), or by covalently linked to a solid support. Alternatively, antibodies can be indirectly linked to a solid support using covalent linkers or by using binding pairs, such as solid supports having bound thereto antibodies, which bind the antibodies of interest, preferably, the Fc regions. Alternatively, the solid support can have bound thereto a reagent, such as avidin or biotin, that binds with an antibody respectively linked to biotin or avidin.

### Disease State or Condition

Disease states or conditions of the present invention can be any disease state or condition that affect only humans. Alternatively, the disease state or condition can affect an animal that has been modified or genetically engineered other than by selective breeding to be an appropriate animal model, or an animal that is an appropriate animal model for the disease state or condition, including animals that have been selectively breed. Disease states of conditions can relate to the structure or function of at least one tissue or organ, such as a tissue or organ that is derived from embryonic tissues from the endoderm, ectoderm or mesoderm.

Disease states or conditions of the present invention include a wide range of pathologies such as are known in the art (Cotran et al., Robbins Pathologic Basis of Disease, 5^{th} Edition, W.B. Saunder Company, Philadelphia (1994), incorporated herein by reference in its entirety). Disease states and conditions have pathological, physical, psychological, cellular, molecular and metabolic components. The present invention is most concerned with identifying antigenic preparations that are related to such disease states or conditions at the pathological, cellular and molecular levels. However, correlation of the present invention with the physical and psychological components of disease states or conditions is an important aspect of the present invention.

Such disease state or conditions include, for example:

1) hemodynamic disorders, thrombosis and shock (such as but not limited to edema, hyperemia and congestion, hemorrhage, hemostatis, thrombosis, embolism, infarction and shock);

2) genetic disorders (such as but not limited to disorders with multifactorial inheritance, normal karyotype cytogenic disorders and single-gene disorders with nonclassic inheritance);

3) diseases of immunity (such as but not limited to immunologic tissue injury, hypersensitivity reactions, autoimmune diseases, immunologic deficiency syndromes and amyloidosis);

4) neoplasia such as cancers, carcinomas, sarcomas, adenocarcinomas, neoplasms, lymphomas, growths and tumors);

5) environmental and nutritional diseases (such as but not limited to chemical and drug injury, physical injuries, protein-calorie under-nutrition, vitamin disorders and deficiencies, and nutritional excesses and imbalances);

6) diseases of infancy and childhood (such as but not limited to birth injuries, congenital malformations, respiratory distress syndrome, erythroblastosis fetalis, inborn errors of metabolism and sudden infant death syndrome);

7) disorders of blood vessels (such as but not limited to endothelial cell dysfunction, vascular smooth muscle cell dysfunction, congenital anomalies, atherosclerosis and other forms of arteriosclerosis, hypertensive and vascular disease, arteriosclerosis, inflammatory disease—vasculitides, Raynaud's disease, aneurysms, disorders of the veins and lymphatics and disorders relating to pathology of therapeutic intervention in vascular disease);

8) disorders of the heart (such as but not limited to congestive heart failure, ischemic heart disease, hypertensive heart disease, valvular heart disease, myocardial diseases, pericardial disease, congenital heart diseases and cardiac transplantation);

9) diseases of red cells and bleeding disorders (such as but not limited to anemias and polycythemia bleeding disorders);

10) diseases of white cells, lymph nodes and spleen (such as but not limited to reactive (inflammatory) proliferations of white cells and nodes, plasma cell dyscreasias and related disorders);

11) diseases and disorders of the lung (such as but not limited to congenital anomalies, atelectasis, diseases of vascular origin, obstructive vs. restrictive pulmonary disease, chronic obstructive pulmonary disease, diffuse interstitial (infiltrative restrictive) diseases, complications of therapies and pleura);

12) disorders of the head and neck (such as but not limited to oral soft tissues, upper airways, ear, neck and salivary glands);

13) disorders of the esophagus (such as but not limited to congenital anomalies, lesions associated with motor dysfunction and varices esophagitis);

14) disorders of the stomach (such as but not limited to stimulation of gastric acid secretion, gastric mucosal protection, congenital anomalies, gastritis and gastric ulceration);

15) disorders of the small and large intestines (such as but not limited to congenital anomalies, vascular disorders, enterocolitis, malabsorption syndromes, idiopathic inflammatory bowel disease and colonic divericulosis bowel obstruction);

16) disorders of the appendix (such as but not limited to appendicitis and acute appendicitis);

17) disorders of the peritoneum (such as but not limited to inflammation);

18) disorders of the liver and biliary tract (such as but not limited to cirrhosis, bilirubin and hepatic bile formation, hepatic failure, inflammatory disorders, drug-induced and toxin-induced liver disease, alcoholic liver disease, inborn errors of metabolism and pediatric liver disease, intrahepatic biliary tract disease, circulatory disorders, hepatic disease associated with pregnancy and transplantation);

19) disorders of the pancreas (such as but not limited to congenital anomalies and pancreatitis);

20) disorders of the kidney (such as but not limited to congenital anomalies, glomerular diseases, diseases affecting tubules and interstitium, diseases of blood vessels, urinary tract obstruction and urolithiasis);

21) disorders of the urinary bladder (such as but not limited to congenital anomalies, inflammations and obstructions);

22) disorders of the male genital system (such as but not limited to the penis, testis and epididymis);

23) disorders of the female genital tract (such as but not limited to the vulva, vagina, cervix, body of uterus and endometrium, fallopian tubes, ovaries and gestational and placental disorders);

24) disorders of the breast (such as but not limited to congenital anomalies, inflammations and fibrocystic disease);

25) disorders of the endocrine system (such as but not limited to the pituitary gland, the thyroid gland, the parathyroid glands, the adrenal cortex, the adrenal medulla, the thymus, the pineal gland);

26) disorders (such as but not limited to disorders of pigmentation and melanocytes, acute inflammatory dermatoses, chronic inflammatory dermatoses, blistering (bullous) disease and infestation);

27) disorders of the skeletal system and soft tissues (such as but not limited to developmental abnormalities, diseases associated with abnormal matrix, diseases associated by asetoclast dysfunction, diseases associated with abnormal mineral homeostasis, fractures, osteonecrosis, avascular necrosis, osteomyelitis and tumor-like lesions);

28) disorders of the joints (such as but not limited to osteoarthritis, rheumatoid arthritis, seronegative spondyloarthropathies, infections arthritis and crystal arthropathies);

29) disorders of the peripheral nerve and skeletal muscle (such as but not limited to diseases of peripheral nerves, diseases of skeletal muscle and diseases of the neuromuscular junction); and

30) disorders of the central nervous system (such as but not limited to common pathophysiologic complications malformations and developmental diseases, perinatal brain injury trauma, cerebrovascular diseases, demyelinating diseases, degenerative diseases, inborn errors of metabolism, toxic and acquired metabolic diseases and neurocutaneous syndromes (phakomatoses)).

Preferred aspects of the present invention include a wide variety of disease states or conditions, such as, for example disease states or conditions that can relate to a cellular proliferative disorder or a cellular non-proliferative disorder, a neurodegenerative disease state or condition, an autoimmune disease state or condition, an ischemic disease state or condition or a trauma disease state or condition.

### Preparation of Disease States or Conditions

Preparations of disease states or conditions can be made using appropriate methods known in the art. For example, appropriate samples of tissues, organs, cells or fluids can be obtained from a human subject having, suspected of having, had or suspected of having had the disease state or condition provide in vivo samples for disease states or conditions. Alternatively, appropriate samples of tissues, organs cells or fluids can be obtained from an animal, such as an appropriate animal model, including animals bred or engineered or otherwise modified to be an animal model for a disease state or condition to provide in vivo samples for such disease states or conditions. Also, cultures of organs or tissues obtained from a human subject or an animal can be kept in culture to provide ex vivo samples for disease states or conditions. Furthermore, in another aspect of the present invention, tissue culture can be used to propagate cells derived from appropriate tissues, organs, cells or fluids that relate to a disease state or condition to provide in vitro samples for disease states or conditions.

Appropriate samples for disease states or conditions are one of choice based on the particular disease state or condition and the associated pathology. Such samples can be obtained using appropriate procedures, such as biopsy, necropsy or harvesting. For example, disease states or conditions which manifest pathology in a given tissue, organ or fluid, the affected tissue or organ is a preferred sample for use in the present invention. For example, for cancers, growths, tumors and the like, a sample of the pathological tissue, organ or fluid is a preferred sample. Also, for disease states, which affect a particular tissue, organ or fluid, a sample of the affected tissue, organ or fluid is preferred.

For in vitro samples for disease states or conditions, a sample can be contacted with an appropriate growth medium and under appropriate conditions, for example temperature and gasses, such that cells can propagate and form a crude preparation relating to a disease state or condition.

For ex vivo samples for a disease state or condition, sample is contacted with an appropriate organ culture or whole tissue culture, such as derived from an animal, human volunteer or human cadaver, and placed under appropriate culture conditions such that cells can propagate or remain viable and form a crude preparation relating to an etiological agent. Alternatively, the ex vivo sample, optionally along with suspending materials and fluids, is the crude sample itself.

For in vivo samples for a disease state or condition, samples from animals, human volunteers or human cadavers can be used to provide or form crude preparations relating to a disease state or condition. Such samples can be obtained using appropriate methods known in the art. For example, a preparation relating to a disease state or condition can include a sample obtained from a subject, such as blood, urine, tissue or organ samples, such as from biopsy (such as from human subjects or animal models), necropsy (such as from animal models) or harvesting (such as from post-mortem human subjects or animal models).

In one particularly preferred aspect of the present invention, a preparation relating to a disease state or condition include antigens relating to or uniquely relating to that disease state or 10 condition in a selected organism, preferably human. Preferably, the preparation includes antigens that are unique to the etiological agent, but that need not be the case. For example, antigens from one disease state or condition may cross react with antigens from another disease state or condition, particularly similar or analogous antigens, particularly when the disease states or conditions are derived from similar tissues, organs or fluids or have a similar basis in molecular or cellular characterization. Furthermore, different disease states or conditions can cause, induce or prompt the same or similar physiological response in a subject, which does not detract from the present invention because the physiological response can be correlated with other tests or clinical indicia of a disease state or condition, such as unique symptoms.

Preferably, a preparation relating to a disease state or condition is taken from a locus of a subject where there is a relatively high probability of obtaining indicia of the disease state or condition, such as antigens. The skilled practitioner recognizes that different disease states or conditions have target locations in a subject, such as particular fluids, solids, tissues or organs, or that a physiological response is greater in a particular fluid, solid, tissue or organ of a subject. The choice of an appropriate sample from which to make a preparation relating to a disease state or condition can thus be made based on the etiological agent in question. For example, a subject having a carcinoma of the gastro-intestinal tract would tend to result in the presence of antigens in blood, serum or plasma, stool, urine and in the tissues of the infected subject. In such cases, appropriate samples can be used to make preparations relating to disease states or condition.

These crude preparations can include cells. Alternatively, these crude preparations can include cell free preparations. The particular characteristics of the crude preparation can be determined by the particular culturing conditions, be they in vitro, ex vivo or in vivo, and can be dictated by the particular biology of the disease state or condition.

Crude preparations can also include supernatants, culture media, or filtrates or washes from cultures in vitro. For example, an in vitro sample can be grown or maintained and the cellular mass separated from the media by an appropriate method, such as filtration or centrifugation, to provide a supernatant or filtrate preparation and a disease state or condition mass preparation. The disease state or condition mass preparation can be further washed with media or buffer of different characteristics to provide additional crude preparations. In one aspect of the present invention, such disease state or condition mass preparations can be washed with solutions of differing ionic strength, polarity or other characteristic such that antigens on or within the disease state or condition mass, such as cells, are stripped away or separated from the disease state or condition mass.

Crude preparations can also include supernatants, culture media, or filtrates or washes from cultures ex vivo or in vivo preparations. For example, ex vivo or in vivo preparations, such as organ cultures or tissue cultures, organs or tissues, fluids or other samples, can be washed, infiltrated, homogenized or otherwise broken up, treated with proteases or other agents to separate tissue, organ or cellular structures, in order to free, suspend or otherwise provide a crude preparation. These preparations can be filtered or centrifuged to separate debris from the fluid portion of the sample to form a solid portion and a fluid portion, each of which being crude preparations. The solid portion can be further washed with media or buffer of different characteristics to provide additional crude preparations. In one aspect of the present invention, the solid portion can be washed with solutions of differing ionic strength, polarity or other characteristic such that antigens on or within the solid portion, such as cells or cellular debris, tissue debris, organ debris or the like are stripped away or separated from the solid portion. In another aspect of the present invention, an organ or tissue can be infiltrated with solutions of differing characteristics, such that the infiltrate is collected. For example, an organ or tissue can be infiltrated with solutions of differing ionic strengths or polarity and the infiltrate collected.

The crude preparations can be used as is, or can be further treated or processed. For example, a crude preparation can be irradiated to inactivate cells in the preparation. A crude preparation can also be treated or processed to lyse or disrupt cells. For example, a French press can be utilized to disrupt cells, as can detergents, enzymes, temperature, freeze-thaw and other biological, mechanical and chemical methods.

Crude preparations can also be further treated or processed to provide partially purified preparations or substantially purified preparations. For example, a crude preparation can be partially purified using non-specific methods of separation or purification, such as size exclusion chromatography, ion exchange chromatography, HPLC, FPLC, hydophobic chromatography, reverse phase chromatography, isoelectric focusing, electrophoresis, SDS-PAGE, non-denaturing PAGE and the like. Also, a crude preparation can be substantially purified using specific methods of separation or purification, such as, for example, affinity chromatography. In this aspect of the present invention, specific binding reactions, such as receptor ligand or antibody-antigen binding can be used to isolate particular moieties, such as antigens. Bound moieties can be recovered using methods known in the art, such as using salt or salt gradients, particularly where affinity chromatography using a solid support, such as in a column, is used.

### Isolating

Once a preparation, such as antigens is bound to a solid support, such as through binding with an antibody or antibodies bound to a solid support, the bound preparation can be recovered using methods known in the art. For example, if a column including a solid support having immobilized antibodies with antigens from a preparation bound thereto, the column can be rinsed or washed to remove non-bound entities. Solutions of different ionic strength, or a salt gradient, the use of chaotropic agents, anti-chaotropic agents or shifts in pH can be used to wash the column to elute antigens bound to the antibodies immobilized on the solid support. Fractions eluted from the column can be collected and screened for desired activity, such as binding with antibody preparations that include or are suspected of including antibodies that bind with an etiological agent. Depending on the character of the solid support, additional separation methods can be used. For example, if the solid support is magnetic in character, then a magnetic field can be used to temporarily localize the magnetic solid support so that the solid support can be washed or rinsed such that unbound material can be removed from the preparation. In this way, a composition including moieties relating to a disease state or condition, such as a human disease state or condition, results.

The composition can be stored in a variety of conditions, such as liquid, frozen, suspended or lyophilized. The particular method of storing the composition is one of convenience and related to the intended use and shelf-life of the composition, and the stability of the composition under a variety of conditions, such as temperature, humidity and freeze-thaw.

Screening of compositions of the present invention for binding with antibodies in an appropriate preparation can use, for example, labeled binding reagents to detect the binding of antigen to antibody through a variety of formats, including competitive assays, sandwich assay, direct assays or indirect assays. One preferred method includes Western Blotting. For example, a composition of the present invention can be immobilized upon a solid support. An antibody preparation from a subject, such as a human, known or suspected of containing antibodies relating to a disease state or condition, can be contacted with the antibodies on the solid support. A second or secondary antibody preparation, such as alkaline phosphatase labeled mouse anti-human antibodies is contacted with the solid support. The secondary labeled mouse anti-human antibodies bound to human antibodies on the solid support, such as bound to human antibodies on the solid support, can be detected using appropriate substrates for the enzyme, such as pro-chromogenic substrates. Conversion of the pro-chromogenic substrate can be converted to a chromogen by a catalytic entity bound to the antibody, indicating that the antigen bound with the antibody. Compositions of the present invention that bind with such antibody preparations are preferred compositions of the present invention and can be provided singly or in combination to form a composition of the present invention.

A variety of detectable labels are available, as are a variety of assay formats and can be used in the present invention. For example, detectable labels include radioactive, chromogenic, fluorescent, luminescent, chemiluminescent and the like. Assay formats that are preferred include sandwich assays, direct assays and indirect assays as they are known in the art. The choice of a particular format can be made by the skilled artisan based on preferences of assay formats, labels, sensitivity and available reagents or kits.

If a solid support is not used in the present invention, antibodies bound with antigen in solution, preferably forming a precipitate that includes antibodies cross-linked with antigen. The precipitate, a crude preparation, can be washed using appropriate buffers and environmental conditions, and solubilized. Antibodies can be separated from antigens using a variety of methods, such as affinity chromatography or size exclusion chromatography, for example. The resulting preparation can be characterized as partially purified or substantially purified, depending on the methods used and the purity of the resulting composition.

### Further Processing

The compositions made using methods of the present invention can be further purified using non-specific purification methods or specific purification methods. For example, non-specific purification methods include, but are not limited to, ion exchange chromatography, size exclusion chromatography, electrophoresis, non-denaturing electrophoresis, denaturing electrophoresis, PAGE, SDS-PAGE, isoelectric focusing, blotting, selective precipitation and centrifugation. Specific purification methods include, but are not limited to, affinity chromatography and immunochromatography. These further processed compositions can be screened for desirable characteristics, such as binding with an appropriate antibody preparation, using screening methods discussed herein.

III. Compositions Relating to Human Etiological Agents or Human Disease States or Conditions

The present invention provides a composition, preferably an antigenic composition, relating to a human etiological agent or a human disease state or condition made at least in part using at least one method of the present invention. One aspect of the present invention is a composition, preferably an antigenic composition, relating to a human etiological agent or a human disease state or condition made at least in part using at least one method of the present invention.

The composition can be provided in any appropriate form, such as, for example, a fluid state, a suspended state, a dried state, a frozen state or a lyophilized state. The particular preferred form by which a composition can be provided can be determined based on the intended use of the composition as well as various physical properties of the composition. For example, if the composition is to be used in a kit, such as a test kit, a lyophilized or dry format would be preferred. Also, certain compositions exhibit different stabilities in different forms or during processing to make various forms. For example, some compositions exhibit a decrease in activity upon lyophylization or during freeze-thaw cycles.

In one preferred aspect of the present invention, the composition can be provided immobilized on a solid support. The composition can be immobilized by any appropriate method, such as to make a reversibly, irreversibly, directly or indirectly immobilized composition. Such immobilization methods are known in the art and described herein.

Encompassed by the present invention are diagnostic and prognostic compositions. Diagnostic compositions are useful in the diagnosis of a past or present infection with an etiological agent or presently or previously of having or developing a disease state or condition. Prognostic compositions are useful in predicting the course of an infection of an etiological agent or a disease state or condition, preferably including end-points for that disease state or condition.

The diagnostic compositions and prognostic compositions of the present invention include at least one composition of the present invention, including antigenic compositions or antibody compositions either alone or in combination. These compositions can be used singly or in combination, either immobilized on a solid support or not immobilized. The compositions are preferably stored under appropriate conditions and in an appropriate form for the particular intended use, stability and form of the composition.

In one aspect of the present invention, the diagnostic composition or prognostic composition is provided separate from a solid support, such as in a liquid state, solid state or lyophilized state. The diagnostic composition or prognostic composition can optionally be provided linked to a detectable label, particularly if the compositions are to be used in specific binding reactions, but that is not a requirement of the present invention.

In one preferred aspect of the present invention a diagnostic composition or a prognostic composition is provided immobilized to a solid support. As discussed herein and as known in the art, methods of immobilizing such compositions on appropriate solid supports are available. Preferably, compositions of the present invention are provided immobilized, preferably irreversibly or reversibly immobilized, at a location or locus on a test strip. Such test strips can be chromatographic, such as immunochromatographic, or can be a “dip” type strip where chromatographic separation and movement of materials and reagents are not required for the operation of the test.

In one preferred aspect of the present invention, a composition of the present invention can be immobilized on a discrete location on a test strip to form a zone. That zone can perform any number of functions, such as a detection zone to detect specific binding reactions or a reagent zone to provide a composition as a reagent for a test. Immunochromatographic test strips are generally known in the art, and are exemplified by, for example, U.S. Pat. No. 5,656,503 to May et al., issued Aug. 12, 1997, U.S. Pat. No. 5,120,643 to Ching et al., issued Jun. 9, 1992, U.S. Pat. No. 4,981,786 to Dafforn et al., issued Jan. 1, 1991, U.S. Pat. No. 4,960,691 to Gordon et al., issued Oct. 2, 1990, U.S. Pat. No. 4,837,169 to Toner, issued Jun. 6, 1989, U.S. Pat. No. 4,837,168 to de Jaeger et al., issued Jun. 6, 1989 and U.S. Pat. No. 4,366,241 to Tom et al., issued Dec. 28, 1982.

The efficacy of a composition of the present invention as a diagnostic or prognostic can be established by testing the composition using an appropriate population of positive and negative control samples. For example, a diagnostic for an etiological agent can be tested using samples from subjects known to be infected or having been infected with an etiological agent and subjects known to be free from such infection or previous infection. Alternative methods of diagnosis can be used to identify appropriate samples and can be used as a baseline data set for such evaluations. Appropriate statistical analysis of the results obtained by such studies can be used to analyze diagnostic capability of the composition, such as a composition on a test strip.

For prognostic applications, similar populations of subjects can be used, but preferably include samples from the same subject over the course of time as an infection by an etiological agent or the course of a disease state or condition progresses, where the clinical progression and status of the subject over time is monitored and recorded as well. In that way, the clinical progression of an infection or disease state or condition can be established and correlated to results obtained using compositions of the present invention or other tests that are available or later developed. Using this background information and data, the progression of a disease state or condition, or the course of an infection with an etiological agent, can be predicted. Appropriate statistical analysis of the results obtained by such studies can be used to analyze prognostic capability of the composition, such as a composition on a test strip.

In another aspect of the present invention, the composition can be provided as a therapeutic. In this aspect of the present invention, a composition is provided in an appropriate form, in an appropriate container, in an appropriate dose and optionally with instructions for use, including dosing, regimes and optionally clinical parameters to be monitored and evaluated for a subject receiving a therapeutic composition of the present invention.

In a further aspect of the present invention, the composition can be provided as a vaccine. In this aspect of the present invention, a composition is provided in an appropriate container, in an appropriate dose and optionally with instructions for use, including dosing, regimes and optionally clinical parameters to be monitored and evaluated for a subject receiving a vaccine composition of the present invention

IV. Methods for Making Antibody Preparations, Hybridomas and Immortalized Cells

The present invention provides a method of making an antibody preparation using a composition of the present invention and a method of making a hybridoma or immortalized cell using a composition of the present invention. One aspect of the present invention is a method of making an antibody preparation, including: providing a composition of the present invention, such as an antigenic composition; administering the composition to a subject; and obtaining a sample from the subject that includes antibodies. The present invention also includes a method of making a hybridoma or immortilized cell, including: providing a composition of the present invention, such as an antigenic composition; administering the composition to a subject; obtaining a sample from the subject that comprises antibody producing cells or their precursors; making a hybridoma or immortalized cell from the antibody producing cells or their precursors.

The present invention also includes a method of making an antibody preparation, that includes: providing a composition of the present invention, such as an antigenic composition of the present invention, administering said composition to a subject; obtaining a sample from said subject that comprises antibodies. Preferably, the subject is a test animal, but can also be a human volunteer. The composition of the present invention is preferably administered to the subject with an adjuvant to stimulate an immune response. The subject can be administered the composition by an appropriate route of administration in an appropriate dose and regime to stimulate a humoral immune response to the composition. Blood or serum or plasma samples from the subject can be obtained and used to screen such samples for the presence of antibodies that bind with the composition of the present invention using methods known in the art, such as by radioimmunoassy, competitive assays, direct assays or ELISAs, for example.

The present invention also includes a method of making a hybridoma or immortalized cell, including: providing a composition of the present invention, such as an antigenic composition, administering said composition to a subject, obtaining a sample from said subject that comprises antibody producing cells or their precursors; making a hybridoma or immortalized cell from said antibody producing cells or their precursors.

Preferably, a composition of the present invention is administered to a subject, preferably a test animal, such as a laboratory animal such as a mouse, in an appropriate dose, in an appropriate media, in an appropriate regime and an appropriate route of administration such that a humoral immune response to the composition of the present invention is stimulated. The presence of antibodies to the composition in a blood or serum or plasma sample collected from the subject can be screened using an appropriate immunoassay format.

Antibody producing cells or their precursors can be obtained from the subject by an appropriate method, such as by mobilizing stem cells in the subject and collecting peripheral blood from the subject or by obtaining spleen tissue from the subject, such as through biopsy or by sacrificing an animal, and obtaining cellular preparations from the spleen sample. The antibody producing cells or their precursors can be fused with another cell line, such as an immortalized cell line, using appropriate methods such as PEG fusions or electrofusions. Alternatively, antibody producing cells or their precursors can be immortalized using appropriate methods, such as infection with an appropriate vector, such as a vector including an oncogene. Clones that produce an antibody that binds with a composition of the present invention can be screened using methods known in the art, such as immunoassay formats such as RIAs or ELISAs. Populations of cells that make desired antibodies such as monoclonal antibodies can be subcloned and further screened until a single clone or a limited number of clones in a culture produce one or a few types of antibodies. The resulting cellular preparation, preferably a clonal population of one cell line, can be used to produce antibody preparations such as monoclonal antibody preparations, in in vitro culture or by the production of ascites fluids. Cells can be stored by freezing or by continuous culturing using methods known in the art.

Antibodies made by these methods can be partially purified or substantially purified using methods known in the art, such as by precipitation with ammonium sulfate or by affinity chromatography. The purified preparations can be stored by appropriate methods, such as by refrigeration, freezing or lyophylization.

V. Antibodies, Antibody Preparations, Hybridomas and Immortalized Cells

The present invention provides an antibody, an antibody preparation, a hybridoma or immortalized cell, or an antibody made by such hybridoma or immortalized cell, using a method of the present invention. One aspect of the present invention is an antibody, an antibody preparation, a hybridoma or immortalized cell, or an antibody made by such hybridoma or immortalized cell, using a method of the present invention.

VI. Diagnostics, Prognostics, Test Strips and Zones

The present invention provides a diagnostic, prognostic, test strip or zone that includes an antigenic composition or antibody composition of the present invention. One aspect of the present invention is diagnostic, a test strip or a zone, such as a zone on a test strip that includes an antigenic composition or antibody composition of the present invention.

The present invention includes a diagnostic or prognostic comprising an antigenic composition or antibody composition of the present invention. The diagnostic or prognostic can include a composition of the present invention reversibly immobilized on a solid support, irreversibly immobilized on a solid support or not immobilized on a solid support. When not so immobilized, the composition can be used as a reagent in a diagnostic method or kit, such as in specific binding reactions that can utilize such compositions. In one aspect of the present invention, the composition can be linked to a detectable label.

When immobilized on a solid support, such as irreversibly immobilized or reversibly immobilized, directly immobilized or indirectly immobilized, the composition can be provided in a variety of formats. For example, if the solid support is in a particulate or bead format, then the solid support can be placed in a column for specific binding reactions. If the solid support is in a sheet format, such as nitrocellulose sheets, then the composition can be used for a variety of assay formats, such as in dot-blot or slot blot applications, particularly for specific binding reactions. If the solid support is a strip, then the strip can be used in a chromatographic type assay, such as a specific binding assay, such as an immunochromatographic type assay.

The composition, whether immobilized on a solid support or not, can be stored under appropriate conditions in an appropriate form for the intended use of the composition. For example, the composition of the present invention can be stored in a liquid state, a solid state such as frozen, lyophilized or in suspension at an appropriate temperature for the intended use of the composition and the stability of the composition.

One preferred aspect of the present invention includes a test strip comprising an antigenic composition or antibody composition of the present invention. The test strip can include a variety of structures, which can be integral to each other or independent structures, such as described in U.S. patent application Ser. No. 09/579,673 to Hudak et al., filed May 26, 2000, or U.S. patent application Ser. No. 09/579,672 to Lin et al., filed May 26, 2000, each of which is incorporated by reference. For example, a test strip can include a sample application zone, a reagent zone a detection zone and an optional control zone. All of these zones can be provided on a single integral test strip, or can be provided as separate structures that are operably linked, such as by being in fluid communication with each other.

A composition of the present invention can be irreversibly immobilized or reversibly immobilized on such a zone, depending on the particular format of the test strip. For example, if the composition is to be used as a mobilizable reagent, then the composition is preferably reversibly immobilized on a reagent zone and the composition is optionally detectably labeled with a detectable label. Compositions of the present invention can be detectably labeled using methods and compositions known in the art. In the alternative, a composition can be irreversibly immobilized at a detection zone, particularly when the composition is an antibody composition.

A variety of formats for test strips can be utilized using compositions of the present invention. For example, immunochromatographic assays known in the art are quite varied, which include competitive assays, direct assays, indirect assays and the like.

One preferred aspect of the present invention includes a zone comprising an antigenic composition or antibody composition of the present invention. Preferably, such a zone is a discrete location on a solid support, such as a test strip or a sheet, but that need not be the case. Such zones can perform any function on the test strip, but are preferably reagent zones, detection zones or control zones. The composition of the present invention can be irreversibly immobilized or reversibly immobilized, or directly or indirectly immobilized, on such a zone. Methods for applying compositions to zones, such as zones including nitrocellulose, fiberglass, glass or other appropriate materials, are known in the art. Methods of immobilizing such compositions on such zones are also known in the art.

VII. Methods for Detecting Antibodies that Bind with Antigenic Preparations Relating to Human Etiological Agents

The present invention provides a method for detecting an antibody that binds with an antigenic preparation relating to a human etiological agent. One aspect of the present invention is a method for detecting an antibody that binds with an antigenic preparation relating to a human etiological agent, including: providing a sample from a subject; providing a composition of the present invention, such as an antigenic composition relating to an etiological agent; contacting the sample with the composition; and detecting the binding of one or more components of the sample with the composition.

### Use of Method

One aspect of the present invention is a method that is diagnostic or prognostic of a present infection with a human etiological agent. Another aspect of the present invention is a method that is diagnostic or prognostic of a prior infection with a human etiological agent.

### Subject

The subject for use in these methods can be any subject, such as a human or animal, but is preferably a human. Preferably, the subject is a subject, such as a human, suspected of being infected with said etiological agent.

### Sample

The sample for use in these methods of the present invention can be from any location or type of sample from a subject, but are preferably are from a tissue, solid organ or fluid of said subject. The choice of sample is dependent upon the biology of the etiological agent or disease state or condition that is of interest. Preferably, the sample is blood or serum or plasma, but the sample can also be prepared from other samples, such as biopsy materials from locations on a subject that exhibit infection with an etiological agent or manifestations of a disease state or condition.

### Composition

In one aspect of the present invention, the composition is provided not immobilized on a solid support. In this aspect of the present invention, the composition can be used in a homogeneous assay format, for example. Preferably, a composition of the present invention is provided on a solid support, such as on a test strip.

VIII. Methods for Detecting Antibodies that Bind with Antigenic Preparations Relating to Human Disease State or Conditions

The present invention provides a method for detecting an antibody that binds with an antigenic preparation relating to a human disease state or condition. One aspect of the present invention is a method for detecting an antibody that binds with an antigenic preparation relating to a human disease state or condition, including: providing a sample from a subject; providing a composition of the present invention, such as an antigenic composition relating to a disease state or condition; contacting the sample with the composition; and detecting the binding of one or more components of the sample with the composition.

### Use of Method

In one aspect of the present invention, the method is diagnostic of a present human disease state or condition or a prior human disease state or condition. In another aspect of the present invention, the method is prognostic of a present human disease state or condition or a prior human disease state or condition.

### Subject

The subject can be any subject, such as an animal or a human, but is preferably a human subject. The subject preferably is suspected of having said human disease state or condition.

### Sample

The sample for use in these methods of the present invention can be from any location or type of sample from a subject, but are preferably are from a tissue, solid organ or fluid of said subject. The choice of sample is dependent upon the biology of the disease state or condition that is of interest. Preferably, the sample is blood or serum or plasma, but the sample can also be prepared from other samples, such as biopsy materials from locations on a subject that exhibit infection with an etiological agent or manifestations of a disease state or condition.

### Composition

In one aspect of the present invention, the composition is provided not immobilized on a solid support. In this aspect of the present invention, the composition can be used in a homogeneous assay format, for example. Preferably, a composition of the present invention is provided on a solid support, such as on a test strip.

IX. Methods for Detecting Antigens that Bind with Antibody Preparations Relating to Human Etiological Agents

The present invention provides a method for detecting an antigen that binds with an antibody preparation relating to a human etiological agent. One aspect of the present invention is a method for detecting an antigen that binds with an antibody preparation relating to a human etiological agent, including: providing a sample; providing a composition of the present invention, such as an antibody preparation of the present invention relating to an etiological agent; contacting the sample with the composition; and detecting the binding of one or more components of the sample with the composition.

### Use of Method

In one aspect of the present invention, the method is diagnostic of a present infection with human etiological agent. In another aspect of the present invention, the method is diagnostic of a prior infection with a human etiological agent.

### Subject

The subject can be any subject, such as an animal or a human, but is preferably a human subject. The subject preferably is suspected of being infected with said etiological agent or suspected of previously being infected with said etiological agent.

### Sample

The sample for use in these methods of the present invention can be from any location or type of sample from a subject, but are preferably are from a tissue, solid organ or fluid of said subject. The choice of sample is dependent upon the biology of the etiological agent that is of interest. Preferably, the sample is blood or serum or plasma, but the sample can also be prepared from other samples, such as biopsy materials from locations on a subject that exhibit infection with an etiological agent or manifestations of a disease state or condition.

### Composition

In one aspect of the present invention, the composition is provided not immobilized on a solid support. In this aspect of the present invention, the composition can be used in a homogeneous assay format, for example. Preferably, a composition of the present invention is provided on a solid support, such as on a test strip.

X. Methods for Detecting Antigens that Bind with Antibody Preparations Relating to Human Disease States or Conditions

The present invention provides a method for detecting an antigen that binds with an antibody preparation relating to a human disease state or condition. One aspect of the present invention is a method for detecting an antigen that binds with an antibody preparation relating to a human disease state or condition, including: providing a sample from a subject; providing a composition of the present invention, such as an antibody of the present invention relating to a disease state or condition; contacting the sample with the composition; and detecting the binding of at least one component of the sample with the composition.

### Use of Method

In one aspect of the present invention, the method is diagnostic of a present human disease state or condition or a prior human disease state or condition. In another aspect of the present invention, the method is prognostic of a present human disease state or condition or a prior human disease state or condition.

### Subject

The subject can be any subject, such as an animal or a human, but is preferably a human subject. The subject preferably is suspected of being infected with said etiological agent or suspected of previously being infected with said etiological agent.

### Sample

The sample for use in these methods of the present invention can be from any location or type of sample from a subject, but are preferably are from a tissue, solid organ or fluid of said subject. The choice of sample is dependent upon the biology of the disease state or condition that is of interest. Preferably, the sample is blood or serum or plasma, but the sample can also be prepared from other samples, such as biopsy materials from locations on a subject that exhibit a manifestation of a disease state or condition.

### Composition

In one aspect of the present invention, the composition is provided not immobilized on a solid support. In this aspect of the present invention, the composition can be used in a homogeneous assay format, for example. Preferably, a composition of the present invention is provided on a solid support, such as on a test strip.

### EXAMPLES

The following examples exemplify the various aspects of the present invention.

### Example 1

### Bacteria: Helicobacter Pylori

This example relates to methods and compositions of the present invention that relate to bacteria, in particular Helicobacter pylori.

### Recovery of Antibodies

Whole blood, serum or plasma is collected from a subject confirmed, by endoscopy or the Urea Breath Test, to have H. pylon infection. If using whole blood, plasma is collected by centrifugation, as is commonly employed in the art. The plasma from three different subjects is pooled and mixed for sixty minutes at 4° C. Ammonium sulfate is added to the plasma pool until its final concentration equaled 40% (w/v). The ammonium sulfate/plasma solution is mixed for an additional thirty minutes at 4° C., then the solution is transferred equally to an even number of 1 liter centrifuge bottles and is allowed to sit at 4° C. for an additional three hours. The bottles are centrifuged at 2,000×g for 30 minutes at 4° C. and the precipitate recovered. The precipitate is dissolved in one-fourth of it original volume with 50 mM PBS, pH 7.4.

### Antibody Purification

One column volume of the antibody preparation solution described in the preceding section is applied to a Protein-G-sepharose affinity column until the solution has all been completely applied to the gel. The column is then washed with five column volumes of 50 mM PBS pH 7.4 and the antibody is recovered by elution with 400 mM Glycine-HCl, pH 3.5. The eluate is monitored for optical density at UV 280 nm and peak fractions are collected at a volume each approximately equal to one-tenth of the column volume. These fractions are neutralized with 1 M Tris buffer (pH 8.0) until their resultant pH 7.0.

ELISA tests to determine the titer of anti-H. pylori antibody are commercially available. High titer anti-H. pylon serum/plasma from infected individuals can be identified. Nevertheless, the percentage of specific anti-H. pylori antibody present in this initial purification from high titer serum can be low. An enrichment or recovery of H. pylon specific antibodies from this purification process can be used to substantially improve the yield of H. pylori antigen.

### Enrichment of H. pylori Specific Antibody

An enrichment procedure may be employed to increase the relative yield of such specific antibody. Cultured H. pylori cells are fixed with formaldehyde by exposing the H. pylon cells to an appropriate concentration of formaldehyde (such as 1% formaldehyde solution in distilled water) for an appropriate amount of time (such as about sixty minutes) at an appropriate temperature (such as refrigerated temperature 4° C.) as is know by those skilled in this art. These fixed bacterial cells are washed with PBS, fragmented with a sonicator or a homogenizer and covalently linked to a solid support such as Sepharose 4B and packed into a column. Enrichment of specific anti-H. pylori antibody can be achieved by passing the purified anti-H. Pylori antibody preparation described above in the preceding sections through this H. pylori-Sepharose 4B column by essentially following the loading, washing and elution procedures as indicated in the preceding sections.

### High Efficiency Purification of H. pylon Antigen with Anti-H. pylori Antibody

Purified anti-H pylori antibody can be covalently linked to a solid support such as Sepharose 4 B or Sepharose 6B and packed into a column as is known by those skilled in the art. H. pylon cells can be fragmented by sonication, homogenization or lyzing (freeze & thaw, chaotropic agents, etc.). Cell fragments or lysates are exposed to solubilizing detergent to obtain soluble antigens and are mixed for 60 minutes at room temperature. This solution is passed through a 0.45 micron filter, diluted 50 fold with 50 mM PBS, pH 7.4 and applied to the anti-H. pylon. Sepharose column. The column is washed with 5 column volumes of PBS. After washing, bound H. pylori antigen can be eluted from the column with 200 mM Glycine-HCl, pH 3.5. The eluate is monitored by optical density at UV 280 nm and peak fractions are collected at a volume each equal to one-tenth of the column volume. These fractions are immediately treated with 1 M Tris buffer, pH 8.0 until their resultant pH 7.0.

### Example 2

### Viruses: Human Hepatitis B Virus

This example relates to methods and compositions of the present invention that relate to viruses, in particular Human Hepatitis B Virus (HBV). HBV is a major cause or viral hepatitis. Not only does it cause acute viral hepatitis, but also cause chronic viral infections in over 100 million people in the world, primarily in Southeast Asia regions. Purification of HBV antigen from human serum is normally through precipitation with polyethylene glycol followed by zonal centrifugation. In the present invention, a new and efficient purification method has been described to purified HBV antigens from human blood, serum or plasma samples.

### Recovery and Purification of Anti-HBV Antibodies

High titer anti-HBV antibodies are recovered from serum samples by ammonium sulfate precipitation, followed by protein G affinity chromatography. Serum from two different subjects are pooled and mixed for sixty minutes at 4° C. Ammonium sulfate is added to the serum pool until its final concentration equaled 40% (w/v). The ammonium sulfate/serum solution is mixed for an additional thirty minutes at 4° C., then the solution is transferred equally to two- 1 liter centrifuge bottles and is allowed to stand at 4° C. for an additional two hours. The bottles are centrifuged at 2,000×g for 30 minutes at 4° C. and the precipitate recovered. The precipitate from both bottles is dissolved in 400 mL of 50 mM PBS, pH 7.4.

This solution is applied to a Protein-G-sepharose column until it has all been completely applied to the gel. The column is then washed with two liters (5 column volumes) of 50 mM PBS pH 7.4. The antibody is recovered by elution with 400 mM Glycine-HCl, pH 3.5. The eluate is monitored by optical density at UV 280 nm and peak fractions are collected at a volume 1/10 that of the affinity column per fraction. These fractions are neutralized with 1 M Tris buffer, pH 8.0 until their resultant pH 7.0.

### Enrichment of Anti-HBV Antibody

Enrichment of the antibody preparation described in the preceding section can be achieved by using affinity chromatography. Relatively large amounts of HBV antigens can be obtained commercially. These HBV antigens can be covalently linked to a solid support such as CNBR-activated Sepharose 4B, and packed into a column using methods described in the art. The antibody preparation described in the preceding section, can be passed through this affinity column. The column is then washed with five column volumes of PBS. After washing, bound HBV antibodies can be eluted from the column with 200 mM Glycine-HCl, pH 3.5. The eluate is monitored at UV 280 and peak fractions are collected at a volume each equal to 1/20 of the column volume. These fractions are immediately treated with 1 M Tris buffer, pH 8.0 until their resultant pH 7.0.

### High Efficiency Purification of HBV Antigen

High efficiency purification of HBV antigens can be achieved by using an immunoaffinity chromatography method. Anti-HBV antibodies from the enriched anti-HBV antibody preparation described in the preceding section can be covalently-linked to a solid support such as Sepharose 6B using methods known in the art. HBV antigens presented in HBV positive blood, serum or plasma samples can be purified by passing the diluted sample through this column. After washing, highly purified HBV viral antigens can be eluted from the column by an appropriate elution buffer, such as a buffer containing salts or gradients of salts. This antigen preparation is suitable for research, diagnostic and medical uses.

### Example 3

### Disease States or Conditions: Cancer

This example relates to methods and compositions of the present invention that relate to disease states or conditions, in particular cancer, such as skin cancer (such as malignant melanoma). Malignant Melanoma is a deadly form of skin cancer. The etiology of melanoma varies based on the country, host factors and environmental factors. Generally, the incidence rates for melanoma appear to be doubling in many countries approximately every ten to fifteen years, where the mortality rates are increasing slightly less rapidly. The incidence of melanoma increase with age (see generally Love et al., Manual of Clinical Oncology, Sixth Edition, Springer-Verlag, New York).

### Purification of Anti-melanoma Antibodies

Anti-melanoma antibodies developed in melanoma patients can be found in blood, serum, plasma, melanoma biopsy materials or lymph node biopsy materials. Anti-melanoma antibodies can be purified by homogenization of tissue parts, filtration followed by ammonium sulfate precipitation and ion-exchange chromatography with DEAE Cellulose and affinity columns. However, quantity of specific anti-melanoma antibody presented in these initial antibody preparations is usually low when comparing it to the total immunoglobulin load in these samples.

### Enrichment of Anti-melanoma Antibody

Enrichment of the initial antibody preparations described in the preceding section can be achieved by using affinity chromatography. Melanoma antigens can be prepared from surgically removed melanoma tissues through a combination of procedures including cell rupture with freeze-thaw, and antigen solubilization with buffers, salts, enzymes, detergents, physical methods, filtrations or combinations thereof. These crude melanoma antigen mixtures can be covalently linked to a solid support such as CNBR-activated Sepharose 4B, and packed into a column. Initial antibody preparations described in the preceding section can be passed through this affinity column. After washing, antibody bound to this affinity column can be eluted from the column using methods known in the art. The improvement or enrichment of the purity and the yield of the anti-melanoma antibodies is therefore achieved.

### High Efficiency Purification of Melanoma Antigens

High efficiency purification of melanoma antigens can be achieved by using an immunoaffinity chromatography method. Enriched anti-melanoma antibody preparations such as those described in the preceding section can be covalently-linked to a solid support such as Sepharose 4B or 6B. Melanoma antigens in the disrupted cell components and solubilized cell fractions derived from surgically removed melanoma tissues or other biopsy materials, in turn, can be purified by passing the extracted cellular preparations (described in the preceding section) through the column. After washing, purified antigens, antigen fragments or related proteins can be eluted from the column by an elution buffer. This antigen preparation is suitable for research, diagnostic and medical uses.

All publications, including patent documents and scientific articles, referred to in this application and the bibliography and attachments are incorporated by reference in their entirety for all purposes to the same extent as if each individual publication were individually incorporated by reference.

All headings are for the convenience of the reader and should not be used to limit the meaning of the text that follows the heading, unless so specified.

What is claimed is:

1. A method for preparing a composition relating to a human etiological agent, comprising:
1. providing at least one preparation of antibodies comprising human antibodies optionally provided on a solid support;
2. providing at least one preparation of at least one human etiological agent;
3. contacting said preparation with said preparation of antibodies;
4. isolating moieties bound to the said preparation of antibodies to provide an isolated composition relating to a human etiological agent.

2. The method of claim 1, wherein said solid support comprises a sheet, bead, particle, polymer, well or column.

3. The method of claim 1, wherein said preparation of antibodies comprises human antibodies from one single subject, or from multiple subjects.

4. The method of claim 1, wherein said preparation of antibodies comprises human antibodies from at least one subject currently or previously infected with, previously suspected of being infected with or previously exhibiting symptoms of infection with said human etiological agent.

5. The method of claim 1, wherein said preparation of antibodies comprises at least one of class of antibodies selected from the group consisting of IgG, IgM, IgE, IgA or IgD.

6. The method of claim 1, wherein said preparation of antibodies is irreversibly immobilized or reversibly immobilized on said solid support.

7. The method of claim 1, wherein said preparation of antibodies are directly immobilized or indirectly immobilized on said solid support.

8. The method of claim 1, wherein said human etiological agent is selected from the group consisting of whole or cell free preparation of bacteria, virus, parasite, fungus and prion or is a product derived from an etiological agent selected from the group consisting of bacteria, virus, parasite, fungus and prion.

9. The method of claim 1, wherein said preparation of at least one human etiological agent comprises a crude preparation, a partially purified preparation or a substantially purified preparation.

10. The method of claim 1, wherein said preparation of at least one human etiological agent comprises an in vitro preparation, an ex vivo preparation and an in vivo preparation.

11. The method of claim 1, wherein said isolation comprises recovering moieties bound to said solid support from at least one preparation of antibodies.

12. The method of claim 1, wherein said isolated composition is further purified using at least one method selected from the group consisting of ion exchange chromatography, affinity chromatography, size exclusion chromatography, electrophoresis, non-denaturing electrophoresis, denaturing electrophoresis, PAGE, SDS-PAGE, isoelectric focusing, blotting, selective precipitation and centrifugation.

13. The composition made of claim 1, wherein said composition is antigenic.

14. The composition of claim 1, wherein said composition is in a fluid state, a suspended state, a dried state, a frozen state or a lyophilized state.

15. The composition of claim 1, wherein said composition is immobilized on a solid support.

16. The composition of claim 1, wherein said composition is a therapeutic composition, a vaccine composition, a diagnostic composition or a prognostic composition.

17. A method for preparing a composition relating to a human disease state or condition, comprising:
1. providing at least one preparation of antibodies comprising human antibodies optionally provided on a solid support;
2. providing at least one preparation of at least one human disease state or condition;
3. contacting said preparation with said preparation of antibodies;
4. isolating moieties bound to said preparation of antibodies to provide an isolated composition relating to a human disease state or condition.

18. The method of claim 17, wherein said solid support comprises a sheet, bead, particle, polymer, well or column.

19. The method of claim 17, wherein said preparation of antibodies comprises human antibodies from a single subject, or pooled multiple subjects.

20. The method of claim 17, wherein said preparation of antibodies comprises human antibodies from at least one subject currently or previously having, suspected of having or exhibiting symptoms of said human disease state or condition.

21. The method of claim 17, wherein said preparation of antibodies comprises at least one of class of antibodies selected from the group consisting of IgG, IgM, IgE, IgA or IgD.

22. The method of claim 17, wherein said preparation of antibodies are directly immobilized or indirectly immobilized on said solid support.

23. The method of claim 17, wherein said preparation of antibodies is irreversibly immobilized or reversibly immobilized on said solid support.

24. The method of claim 17, wherein said human disease state or condition is related to the structure or function of at least one tissue or organ derived from the endoderm, ectoderm or mesoderm.

25. The method of claim 17, wherein said human disease state or condition is a cellular proliferative disorder or cellular non-proliferative disorder.

26. The method of claim 17, wherein said human disease state or condition is a cancer, carcinoma, lymphoma, sarcoma, malignancy, growth or tumor.

27. The method of claim 17, wherein said human disease state or condition is a neurodegenerative disease state or condition, or an autoimmune disease state or condition, or an ischemic disease state or condition, or a trauma disease state or condition.

28. The method of claim 17, wherein said preparation of at least one human disease state or condition comprises whole cells or a cell free preparation.

29. The method of claim 17, wherein said preparation of at least one human disease state or condition comprises cells, tissues, organs, fluids or solids.

30. The method of claim 17, wherein said preparation of at least one human disease state or condition comprises a crude preparation, a partially purified preparation or a substantially purified preparation.

31. The method of claim 17, wherein said preparation of at least one human etiological agent comprises an in vitro preparation, an ex vivo preparation and an in vivo preparation.

32. The method of claim 17, wherein said isolating comprises recovering moieties bound to said solid support.

33. The method of claim 17, wherein said isolating comprises recovering moieties bound to said at least one preparation of antibodies.

34. The method of claim 17, wherein said isolated composition is further purified using at least one method selected from the group consisting of ion exchange chromatography, affinity chromatography, size exclusion chromatography, electrophoresis, non-denaturing electrophoresis, denaturing electrophoresis, PAGE, SDS-PAGE, isoelectric focusing, blotting, selective precipitation and centrifugation.

35. The composition made at least in part of claim 17, wherein said composition is antigenic.

36. The composition made at least in part of claim 17, wherein said composition is in a fluid state, a suspended state, a dried state, a frozen state or a lyophilized state.

37. The composition made at least in part of claim 17, wherein said composition is immobilized on a solid support.

38. The composition made at least in part of claim 17, wherein said composition is a therapeutic composition, a vaccine composition, a diagnostic composition or a prognostic composition.

39. A method of making an antibody preparation, comprising:
1. providing a composition of claim 17;
2. administering said composition to a subject;
3. obtaining a sample from said subject that comprises antibodies.

40. A method of making a hybridoma or immortalized cell, comprising:
1. providing a composition of claim 17;
2. administering said composition to a subject;
3. obtaining a sample from said subject that comprises antibody producing cells or their precursors;
4. making a hybridoma or immortalized cell from said antibody producing cells or their precursors.

41. An antibody preparation made using the method of claim 39.

42. A hybridoma or immortalized cell made using the method of claim 40.

43. An antibody made using a hybridoma or immortalized cell of claim 41.

44. A method for detecting an antibody that binds with an antigenic preparation relating to a human etiological agent, comprising:
1. providing a sample from a subject;
2. providing a composition of claim 1 relating to an etiological agent;
3. contacting said sample with said composition;
4. detecting the binding of one or more components of said sample with said composition.

45. The method of claim 44, wherein said method is diagnostic or prognostic of a present or a prior infection with a human etiological agent.

46. The method of claim 44, wherein said subject is a human suspected of being currently or previously infected with said etiological agent.

47. The method of claim 44, wherein said sample is from a tissue, organ or fluid of said subject.

48. The method of claim 44, wherein said composition is provided on a solid support.

49. The method of claim 44, wherein said composition is provided in a therapeutic composition, a vaccine composition, a diagnostic composition or a prognostic composition.

50. A method for detecting an antibody that binds with an antigenic preparation relating to a human disease state or condition, comprising:
1. providing a sample from a subject;
2. providing a composition of claim 17 relating to a disease state or condition;
3. contacting said sample with said composition;
4. detecting the binding of one or more components of said sample with said composition.

51. The method of claim 50, wherein said method is diagnostic or prognosis of a present or a prior human disease state or condition.

52. The method of claim 50, wherein said subject is a human suspected of having or previously having said human disease state or condition.

53. The method of claim 50, wherein said sample is a sample is from a tissue, organ or fluid of said subject.

54. The method of claim 50, wherein said composition is provided on a solid support.

55. The method of claim 50, wherein said composition is provided in a therapeutic composition, a vaccine composition, a diagnostic composition or a prognostic composition.

56. A method for detecting an antigen that binds with an antibody preparation relating to a human etiological agent, comprising:
1. providing a sample;
2. providing a composition of claim 39 relating to an etiological agent;
3. contacting said sample with said composition;
4. detecting the binding of one or more components of said sample with said composition.

57. The method of claim 56, wherein said subject is a human suspected of currently or previously being infected with said etiological agent.

58. The method of claim 56, wherein said sample is a sample from a tissue, organ or fluid of said subject.

59. The method of claim 56, wherein said composition is provided on a solid support.

60. The method of claim 56, wherein said composition is provided in a therapeutic composition, a vaccine composition, a diagnostic composition or a prognostic composition.

61. A method for detecting an antigen that binds with an antibody preparation relating to a human disease state or condition, comprising:
1. providing a sample from a subject;
2. providing a composition of claim 39 relating to a disease state or condition;
3. contacting said sample with said composition;
4. detecting the binding of at least one component of said sample with said composition.

62. The method of claim 61, wherein said method is either diagnostic or prognostic of a prior or a present human disease state or condition.

63. The method of claim 61, wherein said subject is a human suspected of having or previously having said human disease state or condition.

64. The method of claim 61, wherein said sample is a sample is from a tissue, organ or fluid of said subject.

65. The method of claim 61, wherein said composition is provided on a solid support.

66. The method of claim 61, wherein said composition is provided in wherein said composition is in a therapeutic composition, a vaccine composition, a diagnostic composition or a prognostic composition.
